SEQUENCE LISTING (74 sequences)

SEQ ID NO: 1 - protein, 354 aa, Triticum aestivum
MPMGSSAAMPALPPGFRFHPTDEELIVHYLRRQAASMPSPVPIIAEVNIYKCNPWDLPGKALFGENEWYFFSPRDRKYPNGARPNRAAGSGYWKATGTDKAILSTPANESIGVKKALVFYRGKPPKGVKTDWIMHEYRLTAADNRTTKRRGSSMRLDDWVLCRIHKKCGNLPNFSSSDQEQEHEQESSTVEDSQNNHTVSSPKSEAFDGDGDDHLQLQQFRPMAIAKSCSLTDLLNTVDYAALSHLLLDGAGASSSDAGADYQLPPENPLIYSQPPWQQTLHYNNNNGYVNNETIDVPQLPEARVDDYGMNGDKYNGMKRKRSSGSLYCSQLQLPADQYSGMLIHPFLSQQLHM

SEQ ID NO: 2 - protein, 355 aa, Triticum aestivum
MPMGSSSAAMPALPPGFRFHPTDEELIVHYLGRQAASMPSPVPIIAEVNIYKCNPWDLPGKALFGENEWYFFSPRDRKYPNGARPNRAAGSGYWKATGTDKAILSTPANESIGVKKALVFYRGKPPKGVKTDWIMHEYRLTAADNRTTKRRGSSMRLDDWVLCRIHKKCGNLPNFSSSDQEQEHEQESSTVEDSQNNHTVSSPKSEAFDGDGNDHLQLQQFRPMAIAKSCSLTDLLNTVDYAALSHLLLDGAGASSSDAGADYQLPPENPLIYSQPPWQQTLHYNNNNGYVNNETIDVPQLPEAGVDDYGMNGDKYNGMKRKRSSGSLYCSQLQLPADQYSGMLTHPFLSQQLHM

SEQ ID NO: 3 - protein, 359 aa, Triticum aestivum
MPMGSSSAAMPALPPGFRFHPTDEELIVHYLGRQAASMPSPVPIIAEVNIYKCNPWDLPGKALFGENEWYFFSPRDRKYPNGARPNRAAGSGYWKATGTDKAILSTPANESIGVKKALVFYRGKPPKGVKTDWIMHEYRLTAADNRTTKRRGSSMRLDDWVLCRIHKKCNNLHNFSSSDQEQEHEQESSTTVEDSHNNHTVSSPKSEAFDGDGDDQLQLQQFRPMAIAKSCSLTDLLNTVDYAALSHLLLDGAGAGASSSDAGADYQLPPENPLIYSQPPWQQTLHYNNNNNGYVNIDTIDVPQIPEARVDDYGMNGDRYNGMKRKRSSGSLYCSQLQLPADQYSGMLIHPFLSQQLHM

SEQ ID NO: 4 - protein, 354 aa, Triticum aestivum
MPMGSSAAMPALPPGFRFHPTDEELIVHYLGRQAASMPSPVPIIAEVNIYKCNPWDLPGKALFGENEWYFFSPRDRKYPNGARPNRAAGSGYWKATGTDKAILSTPANESIGVKKALVFYRGKPPKGVKTDWIMHEYRLTAADNRTTKRRGSSMRLDDWVLCRIHKKCGNLPNFSSSDQEQEHEQESSTVEDSQNNHTVSSPKSEAFDGDGDDQLQLQQFRPMAIAKSCSLTDLLNTVDYAALSHLLLDGAGASSSDAGADYQLPPENPLIYSQPPWQQTLHYNNNNGYVNNDTIDVPQLPEAHVDDYGMNGDRYNGMKRKRSSGSLYCSQLQLPADQYSGMLIHPFLSQQLHM

SEQ ID NO: 5 - DNA, 1062 bp, Triticum aestivum
atgccaatgggcagcagcgccgccatgcccgccctccctcccggcttccggttccaccccaccgacgaggagctcatcgtccactacctccgcaggcaggccgcgtccatgcccagccccgtgcccatcatcgccgaggtcaacatctacaagtgcaacccatgggacctccccggcaaagctttgttcggggagaatgagtggtacttcttcagcccccgggatcgcaagtaccccaacggcgcgcgcccgaaccgcgccgccgggtccggctactggaaggccaccggcaccgacaaggccatcctgtccacgccggccaacgagagcatcggggtcaagaaggcgctcgtgttctacaggggcaagccgcccaagggcgtcaagaccgactggatcatgcacgagtaccgcctcaccgcagccgacaaccggaccaccaagcgcagaggatcctccatgaggctggatgactgggtgctgtgtaggatccacaagaagtgcggcaacttgcccaacttctcctcctctgaccaggaacaggagcatgagcaggagagctccaccgtggaggactcgcagaacaaccacaccgtgtcgtcgcccaagtccgaggccttcgacggcgacggcgacgaccacctccagttgcagcagttccgccccatggcgatcgccaagtcgtgctccctcaccgacctgctcaacaccgtcgactacgccgcgctctcgcacctcctcctcgacggcgccggcgcctcgtcgtcggacgccggagcagactaccagctgcctcccgaaaacccactcatctactcgcagcctccatggcaacaaacgctacactataataacaacaacggctacgtgaacaacgagaccatcgacgtgcctcagctacccgaggcgcgcgtagatgactacggcatgaatggcgataagtataacggcatgaagaggaagagatccagcggcagcttgtactgcagccagctgcagctcccagcggatcagtacagcggcatgctgatccatccgttcctcagccagcagctgcacatg

SEQ ID NO: 6 - DNA, 1065 bp, Triticum aestivum
atgccaatgggcagcagcagcgccgccatgcccgccctccctcccggcttccggttccaccccaccgacgaggagctcatcgtccactacctcggcaggcaggccgcgtccatgcccagccccgtgcccatcatcgccgaggtcaacatctacaagtgcaacccatgggacctccccggcaaggccttgttcggggagaatgagtggtacttcttcagcccccgggatcgcaagtaccccaacggcgcgcgccccaaccgcgccgccgggtccggctactggaaggccaccggcaccgacaaggccatcctgtccacgccggccaacgagagcatcggcgtcaagaaggcgctcgtgttctacaggggcaagccgcccaagggcgtcaagaccgactggatcatgcacgagtaccgcctcaccgcagccgacaaccggaccaccaagcgcagaggatcctccatgaggctggatgactgggtgctgtgtaggatccacaagaagtgcggcaacttgcccaacttctcctcctctgaccaggaacaggagcatgagcaggagagctccaccgtggaggactcgcagaacaaccacaccgtgtcgtcgcccaagtccgaggccttcgacggcgacggcaacgaccacctccagttgcagcagttccgccccatggcgatcgccaagtcgtgctccctcaccgacctgctcaacaccgtcgactacgccgcgctctcgcacctcctcctcgacggcgccggcgcctcgtcgtcggacgccggagcagactaccagctgccgccggaaaacccactcatctactcgcagcctccatggcaacaaacgctacactataataacaacaacggctacgtgaacaacgagaccatcgacgtgcctcagctacccgaggcgggggtagatgactacggcatgaatggcgataagtataacggcatgaaaaggaaaagatccagcggcagcttgtactgcagccagctgcagctcccagcggatcagtacagcggcatgctgacccatccgttcctcagccagcagctgcacatg

SEQ ID NO: 7 - DNA, 1077 bp, Triticum aestivum
atgccaatgggcagcagcagcgccgccatgcccgccctccctcccggcttccggttccaccccaccgacgaggagctcatcgtccactacctcggcaggcaggccgcgtccatgcccagccccgtgcccatcatcgccgaggtcaacatctacaagtgcaacccatgggacctccccggcaaggccttgttcggggagaatgagtggtacttcttcagcccccgggatcgcaagtaccccaacggcgcgcgccccaaccgcgccgccgggtccggctactggaaggccaccggcaccgacaaggccatcctgtccacgccggccaacgagagcatcggcgtcaagaaggcgctcgtcttctaccggggcaagccgcccaagggcgtcaagaccgactggatcatgcacgagtaccgcctcaccgccgccgacaaccggaccaccaagcgcagaggatcctccatgaggctggatgactgggtgctgtgtaggatccacaagaagtgcaacaacttgcacaacttctcctcctctgaccaggaacaggagcacgagcaggagagctccaccaccgtggaggactcgcacaacaaccacaccgtgtcgtcgcccaagtcggaggccttcgacggcgacggcgacgaccagctgcagctgcagcagttccgccccatggcgatcgccaagtcgtgctccctcaccgacctgctcaacaccgtcgactacgccgcgctctcgcacctcctcctcgacggcgccggcgccggcgcctcgtcgtcggacgccggagcagactaccagctgccgccggaaaacccgctcatctactcgcagcctccatggcaacaaacgctacactataataacaataacaatggctacgtgaacatcgacaccatcgacgtgcctcagatacccgaggcccgtgtagatgactacggcatgaatggcgataggtacaacggcatgaagaggaagaggtccagcggcagtttgtactgcagccagctgcagctcccggcggatcagtacagcggcatgctgatccatccgttcctcagccagcagctgcacatg

SEQ ID NO: 8 - DNA, 1062 bp, Triticum aestivum
atgccaatgggcagcagcgccgccatgcccgccctccctcccggcttccggttccaccccaccgacgaggagctcatcgtccactacctcggcaggcaggccgcgtccatgcccagccccgtgcccatcatcgccgaggtcaacatctacaagtgcaacccatgggacctccccggcaaggccttgttcggggagaatgagtggtacttcttcagcccccgggatcgcaagtaccccaacggcgcgcgccccaaccgcgccgccgggtccggctactggaaggccaccggcaccgacaaggccatcctgtccacgccggccaacgagagcatcggcgtcaagaaggcgctagtgttctacaggggcaagccgcccaagggcgtcaagaccgactggatcatgcacgagtaccgcctcacagccgccgacaaccggaccaccaagcgcagaggatcctccatgaggctggatgactgggtgctgtgtaggatccacaagaagtgcggcaacttgcccaacttctcctcctctgaccaggaacaagagcatgagcaggagagctccaccgtggaggactcgcagaacaaccacaccgtatcgtcgcccaagtcggaggccttcgacggcgacggcgacgaccagctccagctgcagcagttccgccccatggcgatcgccaagtcgtgctccctcaccgacctgctcaacaccgtcgactacgccgcgctctcgcacctcctcctcgacggcgccggcgcctcgtcgtcggacgccggagcagactaccagctgccgccggaaaacccactcatctactcgcagcctccatggcaacaaacgctacactataacaataacaacggctacgtgaacaacgacaccatcgacgtgcctcagctacccgaggcgcatgtagatgactacggcatgaatggcgataggtataacggcatgaagaggaagagatccagcggcagcttgtactgcagccagctgcagctcccggcggatcagtacagcggcatgctgatccatccgttcctcagccagcagctgcacatg

SEQ ID NO: 9 - protein, 156 aa, Triticum aestivum
LPPGFRFHPTDEELIVHYLRRQAASMPSPVPIIAEVNIYKCNPWDLPGKALFGENEWYFFSPRDRKYPNGARPNRAAGSGYWKATGTDKAILSTPANESIGVKKALVFYRGKPPKGVKTDWIMHEYRLTAADNRTTKRRGSSMRLDDWVLCRIHKK

SEQ ID NO: 10 - protein, 104 aa, Hordeum vulgare
LPPGFRFHPTDEELIVHYLGRQAASMPSPVPIIAEVNIYKCNPWDLPGKALFGENEWYFFSPRDRKYPNGARPNRAAGSGYWKATGTDKAILSTPANESIGVKK

SEQ ID NO: 11 - protein, 162 aa, Oryza sativa
LPPGFRFHPTDEELIVHYLMNQAASVKCPVPIIAEVNIYKCNPWDLPGKALFGENEWYFFSPRDRKYPNGARPNRAAGSGYWKATGTDKSILSTPTSDNIGVKKALVFYKGKPPKGVKTDWIMHEYRLTGTSANSTTTTKQRRASSMTMRLDDWVLCRIHKK

SEQ ID NO: 12 - protein, 162 aa, Oryza sativa
LPPGFRFHPTDEELIVHYLMNQAASIKCPVPIIAEVNIYKCNPWDLPGKALFGENEWYFFSPRDRKYPNGARPNRAAGSGYWKATGTDKSILSTPTSDNIGVKKALVFYKGKPPKGVKTDWIMHEYRLTGTSANNTTTTKQRRASSMTMRLDDWVLCRIHKK

SEQ ID NO: 13 - protein, 160 aa, Sorghum bicolor
LPAGFRFHPTDEELIVHYLMNQAASIPCPVPIIAEVNIYQCNPWDLPAKALFGESEWYFFSPRDRKYPNGARPNRAAGSGYWKATGTDKAIMSTPTGQNIGVKKALVFYGGRPPRGVKTDWIMHEYRLVTADADGKTTAAKRRGGSMRLDDWVLCRIHKK

SEQ ID NO: 14 - protein, 153 aa, Medicago truncatula
LPPGFRFHPTDEELIVHYLCNQATSKPCPASIIPEVDIYKFDPWELPDKSEFEENEWYFFSPRERKYPNGVRPNRATLSGYWKATGTDKAIKSGSKQIGVKKSLVFYKGRPPKGVKTDWIMHEYRLIGSQKQTSKHIGSMRLDDWVLCRIYKK

SEQ ID NO: 15 - protein, 153 aa, Arabidopsis thaliana
LPPGFRFHPTDEELIVYYLRNQTMSKPCPVSIIPEVDIYKFDPWQLPEKTEFGENEWYFFSPRERKYPNGVRPNRAAVSGYWKATGTDKAIHSGSSNVGVKKALVFYKGRPPKGIKTDWIMHEYRLHDSRKASTKRNGSMRLDEWVLCRIYKK

SEQ ID NO: 16 - protein, 154 aa, Solanum tuberosum
LPPGFRFHPTDEELIMYYLRYQATSRPCPVSIIPEIDVYKFDPWELPEKAEFGENEWYFFTPRDRKYPNGVRPNRAAVSGYWKATGTDKAIYSANKYVGIKKALVFYKGKPPKGVKTDWIMHEYRLSDSKSQTYSKQSGSMRLDDWVLCRIYKK

SEQ ID NO: 17 - protein, 154 aa, Solanum demissum; identical to SEQ ID NO: 16

SEQ ID NO: 18 - protein, 153 aa, Lycopersicon esculentum
LPPGFRFHPTDEELIMYYLRYQATSRPCPVSIIPEIDVYKFDPWVLPEKAEFGDNEWYFFTPRDRKYPNGVRPNRAAVSGYWKATGTDKAIYSANKYVGIKKALVFYKGKPPKGVKTDWIMHEYRLSDSKSQTSKQSGSMRLDDWVLCRIYKK

SEQ ID NO: 19 - protein, 153 aa, Mesembryanthemum crystallinum
LPPGFRFHPTDEELIIYYLKNQAMSRPCPVSIIPEVDIYKFDPWQLPDKAEFGENEWYFFTPRDRKYPNGIRPNRATVSGYWKATGTDKAIYSGAKYVGIKKALVFYKGKPPKGIKTDWIMHEYRLTESKHQPIKQHGSMRLDNWVLCRIYKK

SEQ ID NO: 20 - protein, 153 aa, Citrus sinensis
LPPGFRFHPTDEELIVHYLRNQATSRPCPVSIIPEVDIYKFDPWQLPEKAEFGEKEWYFFSPRDRKYPNGTRPNRATVSGYWKATGTDKAIYGGSKYLGVKKALVFYKGRPPKGIKTDWIMHEYRLNDPTRQPYKHNGSMKLDDWVLCRIYKK

SEQ ID NO: 21 - protein, 163 aa, Populus tremula x Populus tremuloides
LPPGFRFHPTDEELVVHYLKKKAASVPLPVTIIAEIDLYKLDPWELPSKATFGEQEWYFFSPRDRKYPNGARPNRAATSGYWKATGTDKPILTSNGAQKVGVKKALVFYGGKPPKGIKTDWIMHEYRLIENSSSPRPPAADSATKKGGSLRLDDWVLCRIYKK

SEQ ID NO: 22 - protein, 158 aa, Glycine max
LPPGFRFHPTDEELILHYLSKKVASIPLPVSIIAEVDIYKLDPWDLPAKATFGEKEWYFFSPRDRKYPNGARPNRAAASGYWKATGTDKTIVTSLQGGAQESVGVKKALVFYKGRPPKGVKTNWIMHEYRLVDNNKPIKLKDSSMRLDDWVLCRIYKK

SEQ ID NO: 23 - DNA, 23 bp, Artificial
  gene promoter region
rrwkmcgtrnnnnnyacgtmayy

SEQ ID NO: 24 - DNA, 24 bp, Artificial
  gene promoter region
rswvktynnnnnnnnyacgwcwct

SEQ ID NO: 25 - protein, 21 aa, Artificial
  motif A of wheat TAC69
LPPGFRFHPTDEELIVHYLXR

SEQ ID NO: 26 - protein, 15 aa, Artificial
  motif B of wheat NAC69
IAEVNIYKCNPWDLP

SEQ ID NO: 27 - protein, 36 aa, Artificial
  motif C of wheat NAC69
EWYFFSPRDRKYPNGARPNRAAGSGYWKATGTDKAI

SEQ ID NO: 28 - protein, 29 aa, Artificial
  motif D of wheat NAC69
IGVKKALVFYRGKPPKGVKTDWIMHEYRL

SEQ ID NO: 29 - protein, 11 aa, Artificial
  motif E of wheat NAC69
DDWVLCRIHKK

SEQ ID NO: 30 - DNA, 21 bp, Artificial Sequence
  primer
gcgaggagtctggtggcaact

SEQ ID NO: 31 - DNA, 20 bp, Artificial Sequence
  primer
aagcagagcacggccggtaa

SEQ ID NO: 32 - DNA, 18 bp, Artificial
  primer
cgttcgtggcgtcgtaca

SEQ ID NO: 33 - DNA, 20 bp, Artificial
  primer
gcgtccagctcctggtacat

SEQ ID NO: 34 - DNA, 22 bp, Artificial
  primer
gttggtgaagacgctgtgctac

SEQ ID NO: 35 - DNA, 24 bp, Artificial
  primer
tgctttctagggacttggaagtgt

SEQ ID NO: 36 - DNA, 20 bp, Artificial
  primer
cccttgaagccaagcaatct

SEQ ID NO: 37 - DNA, 19 bp, Artificial
  primer
gcttctccggctccaattc

SEQ ID NO: 38 - DNA, 21 bp, Artificial
  primer
gagagcttaccggcgaagaag

SEQ ID NO: 39 - DNA, 21 bp, Artificial
  primer
gctctcgcactggaaggagat

SEQ ID NO: 40 - DNA, 23 bp, Artificial
  primer
gtggaccagatcttcttccatga

SEQ ID NO: 41 - DNA, 18 bp, Artificial
  primer
gacgaccgggagcttgtc

SEQ ID NO: 42 - DNA, 22 bp, Artificial
  primer
cgacaacctggactgctacaac

SEQ ID NO: 43 - DNA, 22 bp, Artificial
  primer
tctcgcatcatatagccgattg

SEQ ID NO: 44 - DNA, 21 bp, Artificial
  primer
ggataaggatggcgtgtgttg

SEQ ID NO: 45 - DNA, 21 bp, Artificial
  primer
cgctgcatgtatcggctaatc

SEQ ID NO: 46 - DNA, 21 bp, Artificial
  primer
gaacttatgccgccagatcaa

SEQ ID NO: 47 - DNA, 24 bp, Artificial
  primer
gcatacatgtcataccgagcagtc

SEQ ID NO: 48 - DNA, 22 bp, Artificial
  primer
caatcactgcctcactgcttct

SEQ ID NO: 49 - DNA, 22 bp, Artificial
  primer
gccaattaacgggcgtaagata

SEQ ID NO: 50 - DNA, 20 bp, Artificial
  primer
tgtgacctcctcggtgtcag

SEQ ID NO: 51 - DNA, 25 bp, Artificial
  primer
tcttctcgtatcctatagccgattg

SEQ ID NO: 52 - DNA, 23 bp, Artificial
  primer
tactgatcgctcatgctcagaac

SEQ ID NO: 53 - DNA, 19 bp, Artificial
  primer
gttgttgggcgtggaacac

SEQ ID NO: 54 - DNA, 22 bp, Artificial
  primer
gaggaacctccgtccaagaaat

SEQ ID NO: 55 - DNA, 22 bp, Artificial
  primer
aagaccggcatccaggttacta

SEQ ID NO: 56 - DNA, 20 bp, Artificial
  primer
agcccttcgactcgggatta

SEQ ID NO: 57 - DNA, 22 bp, Artificial
  primer
atcggcgttcttgacatcttct

SEQ ID NO: 58 - DNA, 17 bp, Artificial
  primer
tgcctcccgaaaaccca

SEQ ID NO: 59 - DNA, 22 bp, Artificial
  primer
ttgttcacgtagccgttgttgt

SEQ ID NO: 60 - DNA, 23 bp, Artificial
  primer
aacaatggctacgtgaacatcga

SEQ ID NO: 61 - DNA, 20 bp, Artificial
  primer
aaactgccgctggacctctt

SEQ ID NO: 62 - DNA, 29 bp, Artificial
  primer
catggctagcccgagcagcggcggcgcca

SEQ ID NO: 63 - DNA, 34 bp, Artificial
  primer
cgggatccctgcatctgcagatgattgttcagca

SEQ ID NO: 64 - DNA, 30 bp, Artificial
  oligonucleotide
gagatccgtgcacagtacgtaactgttaca

SEQ ID NO: 65 - DNA, 30 bp, Artificial
  oligonucleotide
gagatccgtgcacagtaaataactgttaca

SEQ ID NO: 66 - DNA, 30 bp, Artificial
  oligonucleotide
gagatctgtgcacagtacgtaactgttaca

SEQ ID NO: 67 - DNA, 30 bp, Artificial
  oligonucleotide
gaggtgtttaatgtttacacgtctctagtg

SEQ ID NO: 68 - DNA, 30 bp, Artificial
  oligonucleotide
gaggcccttaatgtttacacgtctctagtg

SEQ ID NO: 69 - DNA, 32 bp, Artificial
  oligonucleotide
cacttgacacgtgggatccacgtcaccatcct

SEQ ID NO: 70 - DNA, 32 bp, Artificial
  oligonucleotide
gccagcgttctgtttctgtacgtctctctggg

SEQ ID NO: 71 - DNA, 32 bp, Artificial
  oligonucleotide
aatccaccgccataaatctacgtaacaagtca

SEQ ID NO: 72 - DNA, 32 bp, Artificial
  oligonucleotide
ttccgtagtttactggatcacgacacacacac

SEQ ID NO: 73 - DNA, 32 bp, Artificial
  oligonucleotide
gatttgtctcgtaatttacacgtaatctgtgt

SEQ ID NO: 74 - DNA, 32 bp, Artificial; identical to SEQ ID NO: 70
  oligonucleotide

(19) United States
(12) Patent Application Publication
(10) Pub. No.: US 2014/0310832 A1
(43) Pub. Date: Oct. 16, 2014

(54) STRESS TOLERANT WHEAT PLANTS

(71) Applicants: Cathrine Lynne McIntyre, Paddington (AU); Gangping Xue, St Lucia (AU)

(72) Inventors: Cathrine Lynne McIntyre, Paddington (AU); Gangping Xue, St Lucia (AU)

(73) Assignee: Commonwealth Scientific and Industrial Research Organisation, Campbell, ACT (AU)

(21) Appl. No.: 14/008,518

(22) PCT Filed: Mar. 28, 2012
(86) PCT No.: PCT/AU2012/000319

§ 371 Date: Feb. 12, 2014

Related U.S. Application Data
(60) Provisional application No. 61/468,235, filed on Mar. 28, 2011.

(51) Int. Cl. C12N 15/82; A23L 1/10; C08B 30/04

(52) U.S. Cl. CPC C12N 15/8237; C12N 15/8271; C12N 15/8261; C12N 15/8273; C12N 15/8251; C12N 15/8282; C08B 30/046; A23L 1/1041
USPC 800/260; 800/320.3; 800/301; 536/23.6; 435/320.1; 435/419; 800/278; 127/67; 127/29; 426/622; 426/661; 426/549; 426/557; 426/619; 426/627; 426/556; 426/592; 426/589

(57) ABSTRACT

The present invention relates to stress tolerant transgenic wheat plants. In particular, the present invention relates to stress tolerant transgenic wheat plants comprising an exogenous polynucleotide encoding a NAC transcription factor operably linked to a promoter capable of directing expression of the polynucleotide in a cell of the plant.

### FIELD OF THE INVENTION

The present invention relates to stress tolerant transgenic wheat plants. In particular, the present invention relates to stress tolerant transgenic wheat plants comprising an exogenous polynucleotide encoding a NAC transcription factor operably linked to a promoter capable of directing expression of the polynucleotide in a cell of the plant.

### BACKGROUND OF THE INVENTION

Soil water deficit is a major constraint to the growth and productivity of small grain crop species such as wheat and barley. One effective adaptive mechanism of plant species to soil water deficit is to increase the expression levels of stress protection or adaptation genes. Many abiotic stress up-regulated regulatory genes have been reported to have an impact on plant dehydration tolerance and improve grain yield (Xiao et al., 2009; Yang et al., 2010). Some regulatory genes that enhance dehydration tolerance also lead to improved water use efficiency, such as HARDY in Arabidopsis (Karaba et al., 2007) and DREB1A in peanut (Bhatnagar-Mathur et al., 2007).

There are many drought up-regulated transcription factors that have been identified so far from various plant species, including important dryland cereal crops wheat and barley (Xue and Loveridge, 2004; Xue et al., 2006; Kam et al., 2008). Among these drought stress up-regulated transcription factor families, drought-inducible members of the NAC (NAM, ATAF/ATAF2 and CUC2) domain-containing transcription factor family have recently been attracting a wide attention to the plant abiotic stress molecular biologists (Tran et al., 2004; Fujita et al., 2004; Hu et al., 2006; Xue et al., 2006; Nakashima et al., 2007; Hu et al., 2008; Pinheiro et al., 2009; Zheng et al., 2009; Gao et al., 2010; Jeong et al., 2010; Peng et al., 2010). In some instances, over-expression of a drought up-regulated NAC gene in model plant species (Arabidopsis and rice) has been reported to show improved drought and/or salt tolerance (Tran et al., 2004; Fujita et al., 2004; Hu et al., 2006; Nakashima et al., 2007; Hu et al., 2008; Zheng et al., 2009; Gao et al., 2010; Jeong et al., 2010; Takasaki et al., 2010). Although some abiotic stress up-regulated members of the NAC family have detrimental effect on plant growth when they are constitutively over-expressed (Nakashima et al., 2007), at least one example of an improvement of grain yield in transgenic rice over-expressing a drought up-regulated NAC gene in water-limited environments has also been reported (Hu et al., 2006; Jeong et al., 2010).

NAC proteins are plant-specific transcription factors, characterised by the presence of a highly conserved NAC domain at the N-terminus (Ernst et al., 2004; Jensen et al., 2010; Nuruzzaman et al., 2010). This conserved domain is involved in DNA-binding recognition and dimerisation (Ernst et al., 2004; Xue et al., 2006; Jeong et al., 2009). The NAC proteins in plants form a large family with 117 members from Arabidopsis and at least 151 members from rice (Nuruzzaman et al., 2010). Besides being involved in abiotic stress responses, many members of the NAC family are constitutively expressed or developmentally regulated (Souer et al., 1996; Takada et al., 2001; Kikuchi et al., 2000; Duval et al., 2002; Xie et al., 2000, 2002; Nuruzzaman et al., 2010). Genetic studies have shown that Petunia NAM and Arabidopsis CUC genes are involved in shoot meristem development (Souer et al., 1996; Aida et al., 1997). Some members of the NAC family have been shown to be involved in floral morphogenesis (Aida et al., 1997; Sablowski and Meyerowitz, 1998), promoting lateral root development (Xie et al., 2000; Xie et al., 2002; He et al., 2005), interacting with a geminivirus protein (Xie et al., 1999) and inhibiting expression of GA₃ up-regulated genes (Robertson, 2004). Other known roles of NAC proteins that have been reported include their involvement in defence response (Xie et al., 1999; Delessert et al., 2005), plant senescence (Uauy et al., 2006; Balazadeh et al., 2010), cell division (Kim et al., 2006) and cell wall synthesis (Zhong et al., 2007; Zhao et al. 2010). There are some NAC members that are involved in both abiotic and biotic stress responses (Hegedus et al., 2003; He et al., 2005; Nakashima et al., 2007; Mauch-Mani and Flors, 2009; Xia et al., 2010a, 2010b). These data suggest that the NAC family play a role in diverse physiological processes. Most of NAC proteins characterised to date serve as transcriptional activators (Tran et al., 2004; Fujita et al., 2004; Hu et al., 2006; Nakashima et al., 2007; Zheng et al., 2009; Gao et al., 2010; Jeong et al., 2010), but two NAC proteins act as repressors have also been reported (Kim et al., 2007, Hao et al., 2010). A further complication is that drought tolerance studies in model plants such as Arabidopsis rarely translate to commercially important crops (Skirycz et al., 2011).

The root-predominant expression and drought up-regulation of four highly homologous TaNAC69 genes (TaNAC69-1, TaNAC69-2, TaNAC69-3 and TaNAC69-4) from bread wheat (Triticum aestivum) has been reported (Xue et al., 2006). TaNAC69-1 DNA-binding specificity with recognition of two consensus DNA-binding sequences and its functional requirement for five highly conserved NAC sub-domain sequences and homodimerisation were also characterised (Xue, 2005; Xue et al., 2006). However, the biological function of TaNAC69 transcription factors in wheat is currently unknown.

There is a need for the identification of transgenic wheat plants which have an enhanced tolerance to stress conditions.

### SUMMARY OF THE INVENTION

There are a vast number of polypeptides which have been identified as possibly having some association with increasing the tolerance of plants to various different types of stress conditions. One family of such polypeptides is known as the NAC (NAM, ATAF/ATAF2 and CUC2) domain-containing transcription factor family. However, this is an extremely large family of proteins in plants, for example there are known to be at least 117 members from Arabidopsis and at least 151 members from rice. There is also considerable diversity between the types of stresses where individual NAC proteins may be up-regulated, as well as unpredictability regarding which family members can be manipulated, and in what manner, to produce an agronomically useful enhancement in crop yields under stress conditions whilst at the same time not significantly compromising yield under non-stress conditions.

The present inventors have identified means to genetically manipulate wheat to provided enhanced tolerance to stress conditions. Thus, in a first aspect, the present invention provides a transgenic wheat plant comprising an exogenous polynucleotide encoding a polypeptide comprising amino acids having a sequence as provided in any one of SEQ ID NOs: 1 to 4, a biologically active fragment thereof, or an amino acid sequence which is at least 90% identical to any one or more of SEQ ID NOs: 1 to 4, wherein the polypeptide is a NAC transcription factor, and wherein the polynucleotide is operably linked to a promoter capable of directing expression of the polynucleotide in a cell of the plant, and wherein the plant is more tolerant to stress conditions than an isogenic wheat plant lacking the exogenous polynucleotide.

The present inventors have shown that tolerance to stress conditions in wheat can be significantly enhanced by ensuring that the exogenous polynucleotide is produced as a suitable level. Accordingly, in a particularly preferred embodiment, the NAC transcription factor is produced in at least a part of the plant at a level at least about 5 fold or 10 fold or 15 fold or 20 fold higher in the transgenic plant when compared to an isogenic wheat plant lacking the exogenous polynucleotide. Preferably, the part of the plant is roots and/or leaves, and more preferably the NAC transcription factor is expressed from the exogenous polynucleotide operably linked to a plant stress inducible promoter.

The promoter may be a constitutive, tissue specific or an inducible promoter. An example of a tissue specific promoter useful for the invention is a root and/or leaf specific promoter. In a preferred embodiment, the promoter is a plant stress inducible promoter.

In an embodiment, when grown under stress conditions the plant

i) produces at least about 1.25 fold or 1.5 fold or 1.75 fold or 2 fold greater shoot and/or root biomass when compared to an isogenic wheat plant lacking the exogenous polynucleotide,

ii) has greater root length when compared to an isogenic wheat plant lacking the exogenous polynucleotide,

iii) has a better water use efficiency than an isogenic wheat plant lacking the exogenous polynucleotide,

iv) has a greater seed yield when compared to an isogenic wheat plant lacking the exogenous polynucleotide,

v) has enhanced expression of an endogenous gene when compared to an isogenic wheat plant lacking the exogenous polynucleotide, wherein the endogenous gene encodes: xyloglucan endo-transglycosylase, dioxygenase, HPPDase, glyoxalase Ia, glyoxalase Ib, chitinase 3-like, xanthine dehydrogenase, beta-alanine synthase, a ZIM family gene, chitinase 3, wali3-like, saccharopine dehydrogenase-like, or cellulose synthase-like protein CslE, or

vi) has a combination of two or more of the features of i) to v).

Preferred combinations at least include features i) and ii), i) and iii), i) and iv), i) and v), ii) and iii), ii) and iv), ii) and v), iii) and iv), iii) and v), and iv and v).

Preferably, the plant when grown under non-stress conditions, produces about the same seed yield, number and/or weight as an isogenic wheat plant lacking the exogenous polynucleotide.

Examples of stress conditions include, but are not limited to, water limitation, salt stress, heat stress, cold stress, fungal infection, or a combination of two or more thereof. In a preferred embodiment, the stress condition is at least water limitation.

In a preferred embodiment, the wheat plant is Triticum aestivum ssp aestivum or Triticum durum.

Preferably, the plant is homozygous for the exogenous polynucleotide.

In an embodiment, the plant is growing in a field.

In another aspect, the present invention provides a transgenic wheat plant,

wherein the transgenic plant has increased expression of a polypeptide comprising amino acids having a sequence as provided in any one of SEQ ID NOs: 1 to 4, a biologically active fragment thereof, or an amino acid sequence which is at least 90% identical to any one or more of SEQ ID NOs: 1 to 4, wherein the polypeptide is a NAC transcription factor, and wherein the plant is more tolerant to stress conditions than an isogenic wheat plant lacking the exogenous polynucleotide.

In an embodiment, the transcription level of an endogenous gene encoding the NAC transcription factor is up-regulated, for example by the presence of an exogenous promoter element 5′ of the start codon that increases transcription levels of the gene.

In another aspect, the present invention provides a population of at least 100 wheat plants of the invention growing in a field.

In a further aspect, the present invention provides an isolated and/or exogenous polynucleotide comprising nucleotides having

i) a sequence as provided in any one of SEQ ID NOs: 5 to 8,

ii) a sequence which is at least 90% identical to any one or more of SEQ ID NOs: 5 to 8, or

iii) a sequence which encodes a polypeptide comprising amino acids having a sequence as provided in any one of SEQ ID NOs: 1 to 4, a biologically active fragment thereof, or an amino acid sequence which is at least 90% identical to any one or more of SEQ ID NOs: 1 to 4,

wherein the polynucleotide encodes a NAC transcription factor, and wherein the polynucleotide is operably linked to a promoter capable of directing expression of the polynucleotide in a cell of a wheat plant.

In an embodiment, the cell is a root or leaf cell.

Also provided is a vector comprising the polynucleotide of the invention.

In another aspect, the present invention provides a cell comprising the polynucleotide of the invention, and/or the vector of the invention. The cell may be a bacterial cell, preferably an Agrobacterium cell which may be used to prepare a wheat cell or wheat plant according to the invention, or a cell of a plant other than wheat, preferably a cereal plant cell such as a rice cell, barley cell, maize cell or sorghum cell. Preferably, the cell is a cell of a wheat plant.

In another aspect, the present invention provides a method of producing a transgenic wheat plant of the invention, the method comprising the steps of

i) introducing the polynucleotide of the invention and/or a vector of the invention into a cell of a wheat plant,

ii) regenerating a transgenic plant from the cell, and

iii) optionally producing one or more progeny from the transgenic plant, thereby producing the transgenic wheat plant.

In a further aspect, the invention provides a method of producing a progeny wheat plant comprising the steps of,

i) obtaining wheat seed comprising the polynucleotide of the invention,

ii) sowing the seed, and

iii) growing a wheat plant from the seed, thereby producing the progeny wheat plant.

In a further aspect, the present invention provides a method of producing a transgenic wheat plant of the invention, the method comprising the steps of

i) crossing two parental wheat plants, wherein at least one is a transgenic wheat plant of the invention,

ii) screening one or more progeny plants from the cross for the presence or absence of the exogenous polynucleotide, and

iii) selecting a progeny plant which comprise the exogenous polynucleotide, thereby producing the transgenic wheat plant.

Preferably, step iii) comprises selecting progeny plants which are homozygous for the exogenous polynucleotide.

Preferably, step iii) comprises analysing the phenotype of the plant or one or more progeny plants thereof for at least one of the features defined herein such as expression level of the polynucleotide, shoot and/or root biomass, root length, water use efficiency, seed yield, other agronomic performance and/or expression of an endogenous gene, and/or selecting a progeny plant which is more tolerant to stress conditions than an isogenic wheat plant lacking the exogenous polynucleotide. Typically, the selection of step iii) will be on the basis of the results of the screening of step ii).

In an embodiment, the method further comprises the step of analysing the plant for at least one other genetic marker.

In another aspect, the present invention provides a method for enhancing the tolerance of a wheat plant to stress conditions, preferably water limitation, the method comprising genetically manipulating said plant such that the production of a polypeptide comprising amino acids having a sequence as provided in any one of SEQ ID NOs: 1 to 4, a biologically active fragment thereof, or an amino acid sequence which is at least 90% identical to any one or more of SEQ ID NOs: 1 to 4, is increased when compared to a wild-type plant, wherein the polypeptide is a NAC transcription factor.

Also provided is a transgenic wheat plant produced using a method of the invention.

In another aspect, the present invention provides for the use of the polynucleotide of the invention and/or a vector of the invention to produce a transgenic wheat plant.

In yet a further aspect, the present invention provides a method for identifying a transgenic wheat plant of the invention, the method comprising the steps of

i) obtaining a nucleic acid sample from a wheat plant, and

ii) screening the sample for the presence or absence of the exogenous polynucleotide,

wherein presence of the exogenous polynucleotide indicates that the plant is a transgenic wheat plant of the invention.

Preferably, the method further comprises growing the plant under stress conditions and analysing the phenotype of the plant, preferably according to at least one of the features defined herein such as expression level of the polynucleotide, shoot and/or root biomass, root length, water use efficiency, seed yield, other agronomic performance and/or expression of an endogenous gene.

In an embodiment, the method further comprises producing a plant from a seed before step i).

In another aspect, the present invention provides a method of producing seed, the method comprising;

i) growing a wheat plant of the invention,

ii) harvesting the seed from the plant, and

iii) optionally processing the seed into a product which is not capable of germinating.

In a further aspect, the present invention provides a method of enhancing or maintaining the yield of seed from a wheat crop, the method comprising

i) growing in a field a crop of plants of the invention and/or a population of the invention,

ii) harvesting seed from the plants, and

iii) optionally processing the seed into a product which is not capable of germinating.

In an embodiment, of the two above aspects, the crop and/or plant is grown under stress conditions and/or there is the possibility the crop and/or plant will be exposed to stress conditions. Examples of such stress conditions include, but are not limited to, water limitation, salt stress, heat stress, cold stress, fungal infection, or a combination of two or more thereof. Most commonly, the stress is a combination of water stress and heat stress, typically for a period of at least one or two days, for example from 1 to 14 days. It is preferred in these embodiments that the wheat plant has a relative yield index of at least 1.02 or 1.03 or 1.04 or 1.05 or 1.06 or 1.07 or 1.10. When the grain yield of the plants grown in the field is less than about 2.5 tonnes/hectare, the relative yield index is preferably from about 1.02 to about 1.15, i.e an increased yield of 2%-15%. When the grain yield of the plants grown in the field is greater than about 2.5 tomes/hectare, the relative yield index is preferably from about 1.02 to about 1.12, i.e an increased yield of 2%-12%.

Also provided is a seed of a plant of the invention, wherein the seed comprises the exogenous polynucleotide. Preferably the seed is homozygous for the exogenous polynucleotide. In an embodiment, the seed has been processed so that it is no longer able to germinate, for example by milling, grinding, polishing, cracking or treatments such as heat treatment.

In another aspect, the present invention provides a method of producing flour, wholemeal, starch or other product, comprising;

a) obtaining seed of the invention, and

b) processing the seed to produce the flour, wholemeal, starch or other product.

In a further aspect, the present invention provides a product produced from a plant of the invention, or part thereof. In one embodiment, the part is the seed of the invention.

In an embodiment, the product is a food product or beverage product. The product may be the final product or an ingredient used to produce a final product. Examples of food products include, but are not limited to, flour, starch, leavened or unleavened breads, pasta, noodles, animal fodder, breakfast cereals, snack foods, cakes, malt, beer, pastries or foods containing flour-based sauces. Examples of beverage products include, but are not limited to, beer or malt.

In an alternate embodiment, the product is a non-food product. Examples of non-food products include, but are not limited to, films, coatings, adhesives, building materials or packaging materials.

In a further aspect, the present invention provides a method of preparing a food product of the invention, the method comprising mixing seed, or flour, wholemeal or starch from said seed, with another food ingredient.

Also provided is a method of preparing malt, comprising the step of germinating seed of the invention.

In another aspect, provided is the use of a plant of the invention, or part thereof such as a seed, as animal feed, or to produce feed for animal consumption or food for human consumption.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Sequence alignment of wheat TaNAC69 proteins. TaNAC69-1 (AY625682) (SEQ ID NO:1), TaNAC69-2 (DQ022842) (SEQ ID NO:2), TaNAC69-3 (DQ022843) (SEQ ID NO:3), TaNAC69-4 (wheat EST ID: 203331236) (SEQ ID NO:4).

FIG. 2. Alignment of NAC transcription factors. Only one NAC protein per plant species sharing the highest homology and >70% identity with the TaNAC69 NAC domain is presented (SEQ ID NO:9). Hv, Hordeum vulgare NAC (gi: 21182621) (SEQ ID NO:10); Os-I, Oryza sativa indica NAC (AAAA02030644) (SEQ ID NO:11); Os-J, Oryza sativa japonica NAC (gi: 29674907) (SEQ ID NO:12); Sb, Sorghum bicolour NAC (gi: 45949087) (SEQ ID NO:13); Mt, Medicago truncatula NAC (gi: 11910349) (SEQ ID NO:14); At, Arabidopsis thaliana NAP (AJ222713) (SEQ ID NO:15); St, Solanum tuberosum NAC (gi: 21920677) (SEQ ID NO:16); Sd, Solanum demissum NAC (AC154033) (SEQ ID NO:17); Le, Lycopersicon esculentum NAC (gi: 5604792) (SEQ ID NO:18); Mc, Mesembryanthemum crystallinum NAC (gi: 26564003) (SEQ ID NO:19); Cs, Citrus sinensis NAC (gi: 38051081) (SEQ ID NO:20); PtxPt, Populus tremula×Populus tremuloides NAC (gi:24106131) (SEQ ID NO:22); Gm, Glycine max NAC (the sequence derived from three EST sequences gi:17962878, 9987195, 21677753) (SEQ ID NO:23). Ide, identity; Sim, similarity.

FIG. 3. Up-regulation of TaNAC69 genes in salt-stressed and rust-infected wheat plants.

(A) Affymetrix data on the rust-infected distal leaves of a rust-resistant line of T. aestivum genotype cv Thatcher in comparison with non-infected plants (GSE6227). Values are means±SD of three biological replicates.

(B) Affymetrix data on the salt-stressed roots of T. aestivum cv Chinese Spring in comparison with non-stressed plants (E-MEXP-971). Values are means±SD of three biological replicates.

(C) Quantitative RT-PCR data on the salt stressed roots of T. aestivum seedlings cv. Bobwhite in comparison with non-stressed plants. Values are means±SD of three biological replicates.

Values in (A) and (B) are hybridisation signal from Affymetrix Wheat Genome Array data (the probe set for TaNAC69 genes is TAAFFX.122104.1.S1_s_AT, which matches at least 6 out of 11 probes with sequences of TaNAC69-1, TaNAC69-3 and TaNAC69-4).

FIG. 4. Over-expression of TaNAC69-1 in T2 homozygous transgenic wheat lines.

(A) TaNAC69-1 expression cassettes. HvDhn8s promoter (AF343068) is the promoter of the barley Dhn8s gene that is constitutively expressed in barley. HvDhn4s promoter (AF343066) is the promoter of the barley Dhn4s gene that is drought-inducible in barley.

(B) Expression levels of TaNAC69-1 in the leaves of transgenic wheat lines and Bobwhite control under non-stress conditions.

(C) Expression levels of TaNAC69-1 in the leaves and roots (insert) of transgenic wheat lines and Bobwhite under drought stress conditions. Expression data were generated using quantitative RT-PCR.

Values in (B) and (C) are means±SD of three biological replicates. D4:NAC69, HvDhn4:TaNAC69; D8:NAC69, HvDhn8:TaNAC69.

FIG. 5. Transgenic wheat lines (T2) over-expressing TaNAC69-1 under dehydration stress.

(A) HvDhn4s:TaNAC69-1 homozygous transgenic wheat lines produced more shoot and root biomass than Bobwhite under PEG treatment for 2 weeks (PEG concentrations were stepwisely increased from 10% to 18%).

(B) HvDhn8s:TaNAC69-1 homozygous transgenic wheat lines grew better than Bobwhite control in a water use efficiency test. Germinated seedlings with similar shoot length were planted in bottles (one plant per bottle) containing 500 g of University of California Mix and 120 ml of 0.3 g L⁻¹ of Aquasol fertilizer solution. No additional water was added after planting. The photograph was taken 1 month after planting.

FIG. 6. Comparative analysis of grain yield per plant and grain weight between D4:NAC69-L3 homozygous transgenic wheat line (at the T3 stage) and non-transgenic control Bobwhite under water-limited conditions. Plants were grown in a glass room with a limited water supply commenced at the early booting stage and experienced dehydration stress during midday in sunny and hot days. Values are means±SD of 12 plants. Hundred grain weight is the weight per 100 grains. * P=0.05.

FIG. 7. Genes that are significantly up-regulated in HvDhn8s-driven TaNAC69-1 (D8NAC69) over-expression homozygous transgenic wheat lines (at the T2 stage). Plants were grown under well-watered conditions. Expression data were generated using quantitative RT-PCR. Values are means±SD of three biological replicates. Statistical significance of differences between transgenic line and Bobwhite was analysed by Student's t-test and indicated by * (P<0.05). Deh, dehydrogenase.

FIG. 8. Relationships in expression level between TaNAC69 and its co-regulated genes in the roots of control and salt-stressed plants. Data from Affymetrix Wheat Genome Array expression analysis (E-MEXP-971). The E-MEXP-971 dataset was generated from the root samples (n=30) of five genotypes with or without salt treatments. Variation in the expression levels of individual genes in this dataset is attributed to both genotypic difference and salt responsiveness. The probe set for TaNAC69 genes is TAAFFX.122104.1.S1_s_AT, which matches at least 6 out of 11 probes with sequences of TaNAC69-1, TaNAC69-3 and TaNAC69-4). Affymetrix probe sets for TaNAC69 co-regulated genes are: Ta.8571.1.S1_x_at (glyoxalase Ia), Ta. 8571.2. S1_x_at (glyoxalase Ib), Ta.21342.1. S1_x_at (chitinase 3-like), Ta.4800.1. S1_at, (saccharopine dehydrogenase-like), Ta. 14172.1. S1_at (ZIM family protein) and Ta.27265.1. S1_x_at (xanthine dehydrogenase). Correlation coefficient (r) between TaNAC69 and its co-regulated gene transcripts is shown in each illustration.

### KEY TO THE SEQUENCE LISTING

SEQ ID NO:1—Amino acid sequence of wheat NAC69-1 polypeptide.
SEQ ID NO:2—Amino acid sequence of wheat NAC69-2 polypeptide.
SEQ ID NO:3—Amino acid sequence of wheat NAC69-3 polypeptide.
SEQ ID NO:4—Amino acid sequence of wheat NAC69-4 polypeptide.
SEQ ID NO:5—Polynucleotide sequence encoding wheat NAC69-1 polypeptide.
SEQ ID NO:6—Polynucleotide sequence encoding wheat NAC69-2 polypeptide.
SEQ ID NO:7—Polynucleotide sequence encoding wheat NAC69-3 polypeptide.
SEQ ID NO:8—Polynucleotide sequence encoding wheat NAC69-4 polypeptide.
SEQ ID NO:9—Amino acid sequence of wheat NAC69-1 (NAC domain).
SEQ ID NO:10—Amino acid sequence of a Hordeum vulgare NAC domain.
SEQ ID NO:11—Amino acid sequence of a Oryza sativa indica NAC domain.
SEQ ID NO:12—Amino acid sequence of a Oryza sativa japonica NAC domain.
SEQ ID NO:13—Amino acid sequence of a Sorghum bicolour NAC domain.
SEQ ID NO:14—Amino acid sequence of a Medicago truncatula NAC domain.
SEQ ID NO:15—Amino acid sequence of a Arabidposis thaliana NAC domain.
SEQ ID NO:16—Amino acid sequence of a Solanum tuberosum NAC domain.
SEQ ID NO:17—Amino acid sequence of a Solanum demissum NAC domain.
SEQ ID NO:18—Amino acid sequence of a Lycopersicon esculentum NAC domain.
SEQ ID NO:19—Amino acid sequence of a Mesembryanthemum crystallinum NAC domain.
SEQ ID NO:20—Amino acid sequence of a Citrus sinensis NAC domain.
SEQ ID NO:21—Amino acid sequence of a Populus tremula×Populus tremuloides NAC domain.
SEQ ID NO:22—Amino acid sequence of a Glycine max NAC domain.
SEQ ID NO:23—NAC transcription factor gene promoter binding region.
SEQ ID NO:24—NAC transcription factor gene promoter binding region.
SEQ ID NO:25—NAC transcription factor motif A.
SEQ ID NO:26—NAC transcription factor motif B.
SEQ ID NO:27—NAC transcription factor motif C.
SEQ ID NO:28—NAC transcription factor motif D.
SEQ ID NO:29—NAC transcription factor motif E.
SEQ ID NOs 30 to 61—Oligonucleotide primers.
SEQ ID NOs 62 to 74—NAC transcription factor binding sites.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, plant molecular genetics, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Stress Conditions

As used herein, the term “stress conditions” refers to any environmental factor(s) that prevents a wheat plant fulfilling its genetic potential. As shown herein, the plants of the invention have enhanced tolerance to both biotic and abiotic stresses. The stress may be constant or transient (for example, at least 7 or at least 14 or at least 21 days). Examples of stress conditions include, but are not limited to, water limitation (drought), salt stress, heat stress, cold stress, freezing stress, radiation stress, oxidative stress, heavy metal tolerance, fungal infection, bacterial infection, viral infection or a combination of two or more thereof. In a preferred embodiment, the stress condition is at least water limitation (in some instances combined with heat stress). “Drought” is defined herein as the limited availability of water to the plants such that the grain yield in the field is less than 2.5 tonnes/hectare, where water limitation is the main limitation to yield. In Australia, this corresponds to an annual rainfall of about 350 mm per year, although the timing of rainfall and therefore water availability to the crop during the season is also important, both prior to planting, during growth and particularly during the grain-filling period. However, it would be appreciated that even with rainfall of greater than 350 mm per year, some extent of water limitation to growth and yield can and does occur, even when grain yields of about 6-7 tonnes/hectare are achieved.

As used herein, the phrase “more tolerant to stress conditions” or variations thereof are considered relative terms. More specifically, the present inventors have identified means to increase (enhance) the ability of wheat to grow under a variety of sub-optimal conditions such as water limitation and increased salt levels. A transgenic wheat plant with enhanced tolerance to stress conditions is defined as a wheat plant better survival, growth and/or yield characteristics (such as greater shoot and/or root biomass, greater root length, better water use efficiency and/or greater seed yield) when compared to an isogenic wheat plant lacking the exogenous polynucleotide.

As used herein, “relative yield index” is defined as yield of transgenic/yield of non-transgenic under conditions which are water-limiting at least in part. In fact, essentially all wheat grown in Australia under non-irrigated conditions is water limited. The average wheat yield in Australia is 2.5 tonnes/hectare, while wheat can produce 15 tonne/ha. The present invention will help bridge this gap.

In one embodiment, a transgenic wheat plant of the invention has “enhanced tolerance to water limitation” (also referred to as “enhanced tolerance to drought”) when compared to an isogenic wheat plant lacking the exogenous polynucleotide. Water limitation (also referred to as drought) refers to a decrease in water availability to a plant that, especially when prolonged (for example for more than 14 days) but also for shorter periods of time (for example for 2-14 days) in particular if combined with another stress such as heat stress (>37° C.), can cause damage to the plant or prevent its successful growth (e.g., limiting plant growth or seed yield). “Drought tolerance” or “tolerance to water limitation” is a trait of a plant to survive under drought conditions over prolonged periods of time without exhibiting substantial physiological or physical deterioration, or to exhibit greater survival or less deterioration than a wild-type (control) plant. “Increased drought tolerance” of a plant is measured relative to a reference or control plant, and is a trait of the plant to survive or have improved probability of surviving under drought conditions over prolonged periods of time, without exhibiting the same degree of physiological or physical deterioration relative to the reference or control plant grown under similar drought conditions. Typically, when a transgenic plant comprising an exogenous polynucleotide in its genome exhibits increased drought tolerance relative to a reference or control plant, the reference or control plant does not comprise in its genome the exogenous polynucleotide.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color, leaf area size, biomass, dry matter or leaf senescence. Other techniques for evaluating drought tolerance include measuring chlorophyll fluorescence, photosynthetic rates and gas exchange rates. The Examples below describe some representative protocols and techniques for simulating drought conditions and/or evaluating drought tolerance.

In another embodiment, a transgenic wheat plant of the invention has “enhanced tolerance to saline and/or sodic soils” when compared to an isogenic wheat plant lacking the exogenous polynucleotide. A saline soil is defined as having a high concentration of soluble salts, high enough to affect plant growth. Salt concentration in a soil is measured in terms of its electrical conductivity. As used herein a “saline soil” has an ECₑ of at least 1 dS/m, more preferably at least 2 dS/m, more preferably at least 3 dS/m, and even more preferably at least 4 dS/m. ECₑ is the electrical conductivity of the ‘saturated paste extract’, that is, of the solution extracted from a soil sample after being mixed with sufficient water to produce a saturated paste. Sodic soils have a low concentration of soluble salts, but a high percent of exchangeable Na⁺; that is, Na⁺ forms a high percent of all cations bound to the negative charges on the clay particles that make up the soil complex. Sodicity is defined in terms of the threshold ESP (exchangable sodium percentage) that causes degradation of soil structure. As used herein a “sodic soil” has an ESP greater than 5, more preferably an ESP greater than 7, more preferably an ESP greater than 9, more preferably an ESP greater than 11, more preferably an ESP greater than 13, and even more preferably an ESP greater than 15. A wheat plant with enhanced tolerance to saline and/or sodic soils is defined as a plant which is more capable of growing, and/or reproducing, in saline and/or sodic conditions when compared to a plant with the same, or similar, genotype but lacking the exogenous polynucleotide.

In a further embodiment, a transgenic wheat plant of the invention has enhanced tolerance to an infection such as by a fungus, bacteria or virus. In a preferred embodiment, a transgenic wheat plant of the invention has enhanced tolerance to a fungal infection. Examples of such fungal infections include, but are not limited to, those by Fusarium graminearum (which causes head blight), Erysiphe graminis f. sp. tritici (which causes powdery mildew). Bipolaris sorokiniana (which causes spot blotch), Puccinia graminis f. sp. tritici (which causes stem rust), Puccinia striiformis (which causes stripe rust) and Puccinia recondita f sp. tritici (which causes leaf rust).

The present invention is predicated, in part, on the discovery that modification of a specific NAC transcription factor in wheat plants alters the production capacity of the plant. In some embodiments, this leads to increased production potential as seen in attributes such as, without limitation, enhanced or improved, maintained or maximized: plant biomass, shoot biomass, plant vigour, seedling vigour, growth rate, height, length and/or width of flag leaf, 1^{st}, 2^{nd} or 3^{rd} true leaves, total leaf area, photosynthetic rate per leaf area, number of leaves per plant, number of heads per plant, average weight of heads per plant, number of tillers per plant, number of seeds per plant, number of seeds per head, average seed weight, total seed weight per plant, starch content or composition of seeds, stem thickness, number of internodes, disease resistance, root mass, number of roots, length of roots, and/or yield and/or delayed senescence compared to a control plant.

In an embodiment, when grown under stress conditions, for example water limitation, a plant of the invention produces at least about 1.25 fold or 1.5 fold or 1.75 fold or 2 fold greater shoot and/or root biomass when compared to an isogenic wheat plant lacking the exogenous polynucleotide. This may be assessed, for example, by exposing the plants to polyethylene glycol induced dehydration experiments for a suitable period of time (for example 2 weeks) starting with 5 day old seedlings in a glasshouse and measuring shoot and root biomass.

In another embodiment, when grown under stress conditions, for example water limitation, a plant of the invention has greater root length when compared to an isogenic wheat plant lacking the exogenous polynucleotide. In an embodiment, the average root length of a plant of the invention is at least 1.2 fold or at least 1.5 fold greater than an isogenic wheat plant lacking the exogenous polynucleotide. This many be assessed, for example, by exposing the plants to polyethylene glycol induced dehydration experiments for a suitable period of time (for example 2 weeks) starting with 5 day old seedlings in a glasshouse and measuring root length.

In a further embodiment, when grown under stress conditions, for example water limitation, a plant of the invention has a better water use efficiency than an isogenic wheat plant lacking the exogenous polynucleotide. This may be assessed, for example, by growing plants in a controlled environment, such as a glasshouse, with a defined amount of water and plastic covering the soil to prevent water loss through evaporation. In an embodiment, biomass is assessed at the early vegetative stage with plants of the invention having a greater biomass than the control plants.

In a further embodiment, when grown under stress conditions, for example water limitation, a plant of the invention has a greater seed yield when compared to an isogenic wheat plant lacking the exogenous polynucleotide. This many be assessed, for example, by growing plants in a controlled environment, such as a glasshouse, with a defined amount of water and plastic covering the soil to prevent water loss through evaporation. In an embodiment, water limitation is commenced at the early booting stage with plants of the invention having a greater grain (seed) yield per plant than the control plants.

In a further embodiment, when grown under stress conditions, for example water limitation, the expression of two or more endogenous genes in a plant of the invention is enhanced when compared to an isogenic wheat plant lacking the exogenous polynucleotide, wherein the endogenous gene encodes; xyloglucan endo-transglycosylase, dioxygenase, HPPDase, glyoxalase Ia, glyoxalase Ib, chitinase 3-like, xanthine dehydrogenase, beta-alanine synthase, a ZIM family gene, chitinase 3, wali3-like, saccharopine dehydrogenase-like, or cellulose synthase-like protein CslE. Details of these genes are summarized in Table 7. Expression of these genes can be detected using standard procedures in the art, for example RT-PCR using the primers outlined in Table 2.

### Polypeptides/Peptides

By “substantially purified polypeptide” or “purified polypeptide” we mean a polypeptide that has generally been separated from the lipids, nucleic acids, other peptides, and other contaminating molecules with which it is associated in its native state. Preferably, the substantially purified polypeptide is at least 90% free from other components with which it is naturally associated.

The term “recombinant” in the context of a polypeptide refers to the polypeptide when produced by a cell, or in a cell-free expression system, in an altered amount or at an altered rate compared to its native state. Typically, the cell comprises a non-endogenous gene that causes an altered amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components. In an embodiment, a “recombinant polypeptide” is a polypeptide made by the expression of a recombinant polynucleotide in a wheat plant cell.

The terms “polypeptide” and “protein” are generally used interchangeably.

As used herein, the term “NAC transcription factor” refers to a (NAM, ATAF/ATAF2 and CUC2) domain-containing transcription factor. NAC transcription factors useful for the invention are also referred to as NAC69 transcription factors. NAC proteins are plant-specific transcription factors, characterised by the presence of a highly conserved NAC domain at the N-terminus (Ernst et al., 2004; Jensen et al., 2010; Nuruzzaman et al., 2010). This conserved domain is involved in DNA-binding recognition and dimerisation (Ernst et al., 2004; Xue et al., 2006; Jeong et al., 2009). A NAC transcription factor useful for the invention comprises amino acids having a sequence as provided in any one of SEQ ID NOs: 1 to 4, a biologically active fragment thereof, or an amino acid sequence which is at least 90% identical to any one or more of SEQ ID NOs: 1 to 4. More preferably, a NAC transcription factor useful for the invention comprises amino acids having a sequence as provided in SEQ ID NO: 1, a biologically active fragment thereof, or an amino acid sequence which is at least 90% identical to SEQ ID NO: 1. In a further preferred embodiment, a NAC transcription factor useful for the invention binds one, preferably both, of the following sites in the promoter region of a gene, rrwkmCGTrnnnnnyACGtmayy (SEQ ID NO: 23) and rswvktynnnnnnnnYACGwcwct (SEQ ID NO: 24) (k=gt, m=ac, r=ag, s=cg, v=acg, w=at, y=ct). Examples of genes, or types of genes, whose transcription is upregulated by a NAC transcription factor useful for the invention (either directly or indirectly), include, but are not limited to, those which encode encodes; xyloglucan endo-transglycosylase, dioxygenase, HPPDase, glyoxalase Ia, glyoxalase Ib, chitinase 3-like, xanthine dehydrogenase, beta-alanine synthase, a ZIM family gene, chitinase 3, wali3-like, saccharopine dehydrogenase-like, or cellulose synthase-like protein CslE. In particular, as shown herein genes which are directly upregulated by a NAC transcription factor useful for the invention include, but are not limited to, class I chitinase, a glyoxalase family 1 member and a ZIM protein family member.

The % identity of a polypeptide is determined by GAP (Needleman and Wunsch, 1970) analysis (GCG program) with a gap creation penalty=5, and a gap extension penalty=0.3. The query sequence is at least 150 amino acids in length, and the GAP analysis aligns the two sequences over a region of at least 150 amino acids. More preferably, the query sequence is at least 250 amino acids in length, and the GAP analysis aligns the two sequences over a region of at least 250 amino acids. More preferably, the query sequence is at least 350 amino acids in length and the GAP analysis aligns the two sequences over a region of at least 350 amino acids. Even more preferably, the GAP analysis aligns two sequences over their entire length.

As used herein a “biologically active” fragment is a portion of a polypeptide of the invention which maintains a defined activity of the full-length polypeptide such as conferring enhanced tolerance to stress conditions to a wheat plant through action as a transcription factor. Biologically active fragments can be any size as long as they maintain the defined activity but are preferably at least 300 or at least 350 amino acid residues long. Preferably, the biologically active fragment maintains at least 10% of the activity of the full length protein.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides useful for the present invention can be prepared by introducing appropriate nucleotide changes into a polynucleotide encoding the polypeptide. Such mutants include, for example, deletions, insertions or substitutions of residues within the amino acid sequence. A combination of deletion, insertion and substitution can be made to arrive at the final construct, provided that the final peptide product possesses the desired characteristics. Preferred amino acid sequence mutants have only one, two, three, four or less than 10 amino acid changes relative to the reference wildtype polypeptide.

Mutant (altered) peptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides useful for the invention are subjected to DNA shuffling techniques as broadly described by Harayama (1998). Products derived from mutated/altered DNA can readily be screened using techniques described herein to determine if they possess NAC transcription factor activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

In a preferred embodiment a mutant/variant polypeptide has one or two or three or four conservative amino acid changes when compared to a naturally occurring polypeptide, or up to 10 or 15 or 20 amino acid changes relative to a reference sequence such as, for example, SEQ ID NO: 1. Details of conservative amino acid changes are provided in Table 1. As the skilled person would be aware, such minor changes can reasonably be predicted not to alter the activity of the polypeptide when expressed in a recombinant cell.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

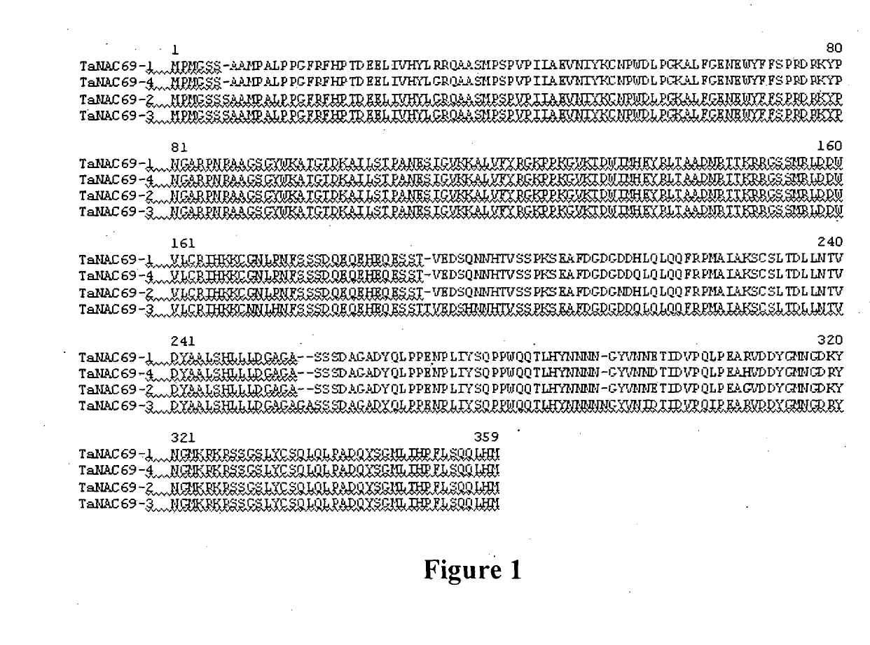

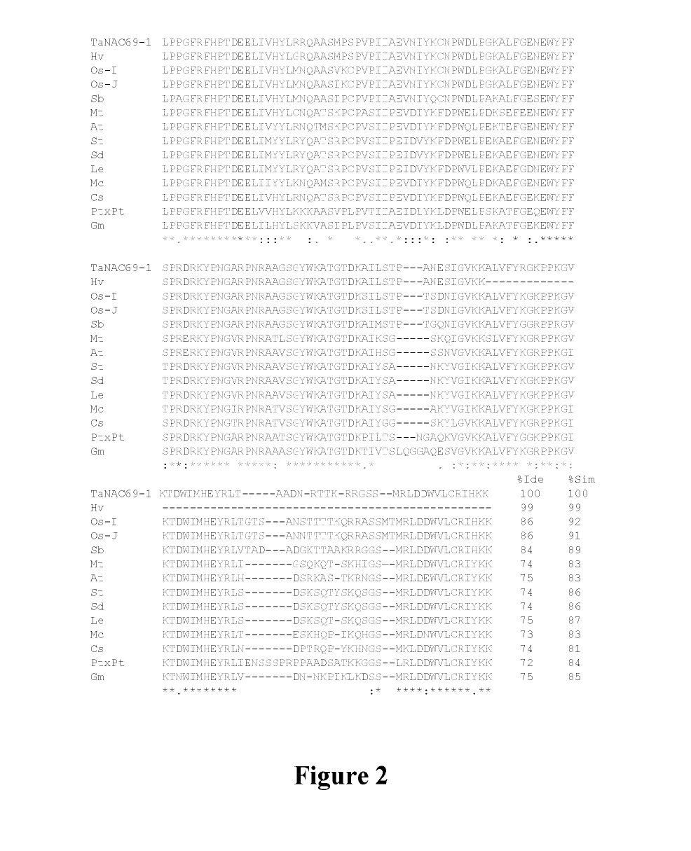

The primary amino acid sequence of NAC transcription factors can be used to design variants/mutants thereof based on comparisons with closely related molecules (see FIGS. 1 and 2). As the skilled addressee will appreciate, residues highly conserved amongst closely related NAC transcription factors are less likely to be able to be altered, especially with non-conservative substitutions, and activity maintained than less conserved residues. In a preferred embodiment, the changes are not in one or more of the motifs which are highly conserved between the different NAC transcription factors, particularly those known to as motifs A, B, C, D and E (Xue et al., 2006; Tran et al., 2010). In a preferred embodiment, these motifs comprise the following sequences;

(SEQ ID NO: 25)
LPPGFRFHPTDEELIVHYL(G/R)R (motif A),
(SEQ ID NO: 26)
IAEVNIYKCNPWDLP (motif B),
(SEQ ID NO: 27)
EWYFFSPRDRKYPNGARPNRAAGSGYWKATGTDKAI (motif C),
(SEQ ID NO: 28)
IGVKKALVFYRGKPPKGVKTDWIMHEYRL (motif D),
and
(SEQ ID NO: 29)
DDWVLCRIHKK (motif E).

Preferably, the polypeptide comprises at least two, more preferably all, of the above motifs.

If one or more of the motifs has a mutation, it is preferred that altered amino acid is a conservative change (see Table 1) and/or can be found in a corresponding position of at least one related protein, such as those provided in FIG. 2.

Also included within the scope of the invention are polypeptides of the present invention which are differentially modified in vitro or in vivo during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, which may be useful in assays for determining the level of NAC transcription factor expression levels or binding to antibodies etc. The polypeptides may be post-translationally modified in a cell, for example by phosphorylation, which may modulate its activity. These modifications may serve to increase the stability and/or bioactivity of the polypeptide of the invention.

### Polynucleotides and Genes

The present invention refers to various polynucleotides. As used herein, a “polynucleotide” or “nucleic acid” or “nucleic acid molecule” means a polymer of nucleotides, which may be DNA or RNA or a combination thereof, and includes mRNA, cRNA, cDNA, tRNA, siRNA, shRNA and hpRNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. An example of a partly-double stranded RNA molecule is a hairpin RNA (hpRNA), short hairpin RNA (shRNA) or self-complementary RNA which include a double stranded stem formed by basepairing between a nucleotide sequence and its complement and a loop sequence which covalently joins the nucleotide sequence and its complement. Basepairing as used herein refers to standard basepairing between nucleotides, including G:U basepairs. “Complementary” means two polynucleotides are capable of basepairing (hybridizing) along part of their lengths, or along the full length of one or both. A “hybridized polynucleotide” means the polynucleotide is actually basepaired to its complement. The term “polynucleotide” is used interchangeably herein with the term “nucleic acid”.

By “isolated polynucleotide” we mean a polynucleotide which has generally been separated from the polynucleotide sequences with which it is associated or linked in its native state. Preferably, the isolated polynucleotide is at least 90% free from other components with which it is naturally associated.

The present invention involves modification of gene activity and the construction and use of chimeric genes. As used herein, the term “gene” includes any deoxyribonucleotide sequence which includes a protein coding region or which is transcribed in a cell but not translated, as well as associated non-coding and regulatory regions. Such associated regions are typically located adjacent to the coding region or the transcribed region on both the 5′ and 3′ ends for a distance of about 2 kb on either side. In this regard, the gene may include control signals such as promoters, enhancers, termination and/or polyadenylation signals that are naturally associated with a given gene, or heterologous control signals in which case the gene is referred to as a “chimeric gene”. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

A genomic form or clone of a gene containing the transcribed region may be interrupted with non-coding sequences termed “introns” or “intervening regions” or “intervening sequences.” An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or for integration into the host genome.

As used herein, a “chimeric gene” refers to any gene that is not a native gene in its native location. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. The term “endogenous” is used herein to refer to a substance that is normally present or produced in an unmodified plant at the same developmental stage as the plant under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism, native genes introduced into a new location within the native host, or chimeric genes. A “transgene” is a gene that has been introduced into the genome by a transformation procedure. The term “genetically modified” includes introducing genes into cells by transformation or transduction, artificially mutating genes in cells and artificially altering or modulating the regulation of a gene in a cell or organisms to which these acts have been done or their progeny.

Furthermore, the term “exogenous” in the context of a polynucleotide (nucleic acid) refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. The query sequence is at least 450 nucleotides in length, and the GAP analysis aligns the two sequences over a region of at least 450 nucleotides. Preferably; the query sequence is at least 750 nucleotides in length, and the GAP analysis aligns the two sequences over a region of at least 750 nucleotides. Even more preferably, the query sequence is at least 1,000 nucleotides in length and the GAP analysis aligns the two sequences over a region of at least 1,000 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In a further embodiment, the present invention relates to polynucleotides which are substantially identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

Polynucleotides useful for the present invention include those which hybridize under stringent conditions to one or more sequences provided as SEQ ID NO's: 5 to 8. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides useful for the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

### Nucleic Acid Constructs

The present invention includes nucleic acid constructs comprising the polynucleotides useful for the invention, and vectors and host cells containing these, methods of their production and use, and uses thereof. The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PolIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of an organism such as a plant. The term constitutive as used herein does not necessarily indicate that a gene is expressed at the same level in all cell types, but that the gene is expressed in a wide range of cell types, although some variation in level is often detectable.

In a preferred embodiment, if a constitutive promoter is used for the invention it results in high levels of mRNA transcribed from the exogenous polynucleotide such that the level of a specific NAC transcription factor that is produced in at least a part of the plant is at least about 5 fold or 10 fold or 15 fold or 20 fold higher when compared to an isogenic wheat plant lacking the exogenous polynucleotide. Non-limiting methods for assessing promoter activity are disclosed by Medberry et al. (1992, 1993), Sambrook et al. (1989, supra) and U.S. Pat. No. 5,164,316. Examples of constitutive promoters which may result in these levels of mRNA production include, but are not limited to, the core promoter of the Rsyn7 promoter and other constitutive promoters disclosed in WO99/43838 and U.S. Pat. No. 6,072,050; the core CaMV 35S promoter (Odell et al., 1985) or its enhanced versions; rice actin (McElroy et al., 1990); ubiquitin (Christensen et al., 1989 and 1992); pEMU (Last et al., 1991); MAS (Velten et al., 1984); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

“Selective expression” as used herein refers to expression almost exclusively in specific organs of, for example, the plant, such as, for example, endosperm, embryo, leaves, or root. In a preferred embodiment, a promoter is expressed selectively or preferentially in leaves and/or roots of a wheat plant. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages. Compartmentation in specific subcellular locations such as the plastid, cytosol, vacuole, or apoplastic space may be achieved by the inclusion in the structure of the gene product of appropriate signals, eg. a signal peptide, for transport to the required cellular compartment, or in the case of the semi-autonomous organelles (plastids and mitochondria) by integration of the transgene with appropriate regulatory sequences directly into the organelle genome.

A “tissue-specific promoter” or “organ-specific promoter” is a promoter that is preferentially expressed in one tissue or organ relative to many other tissues or organs, preferably most if not all other tissues or organs in, for example, a plant. Typically, the promoter is expressed at a level 10-fold higher in the specific tissue or organ than in other tissues or organs.

Tissue-specific promoters useful for the invention include root preferred promoters, such as the maize NAS2 promoter, the maize Cyclo promoter (US 2006/0156439), the maize ROOTMET2 promoter (WO05063998), the CR1BIO promoter (WO06055487), the CRWAQ81 promoter (WO05035770) and the maize ZRP2.47 promoter (NCBI accession number: U38790; GI No. 1063664).

In another embodiment, the promoter is at least capable of expressing the polypeptide in leaves of the plant, particularly developing leaves. Examples of leaf-specific promoters which can be used include those described in Yamamoto et al. (1994 and 1997), Kwon et al. (1994), Gotor et al. (1993), Orozco et al. (1993), Matsuoka et al. (1993) and Stockhaus et al. (1987 and 1989).

“Inducible promoters” selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, infection or drought, phytohormones, wounding, or chemicals such as ethanol, jasmonate, salicylic acid, or safeners. As used herein, a “plant stress inducible promoter” is any inducible promoter that is functional in a wheat plant, and hence this term is not limited to promoters derived from a plant.

Suitable inducible promoters for use in expressing the above-described nucleic acids in a plant include promoters that are induced by drought, ABA, salinity, heat or other stresses often associated with water limitation. Inducible promoters useful for the invention include the following: the Adh1 promoter which is inducible by hypoxia or cold stress, the Hsp70 promoter which is inducible by heat stress, the PPDK promoter which is inducible by light, the Zea mays RAB17 drought inducible promoter induced by ABA (Vilardell, et al., 1990; Busk et al., 1997), the barley drought inducible promoter HvDhn4s (Xiao and Xue, 2001), and the DREB2A and DREB2B promoters induced by dehydration (Liu et al., 1998). Additional drought inducible promoters are disclosed in U.S. Pat. No. 7,314,757.

In a preferred embodiment, the plant stress inducible promoter is a drought inducible promoter, examples of which are provided above.

Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present invention may comprise a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Transcription termination sequences which do not include a polyadenylation signal include terminators for Poll or PolIII RNA polymerase which comprise a run of four or more thymidines. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

As the DNA sequence inserted between the transcription initiation site and the start of the coding sequence, i.e., the untranslated 5′ leader sequence (5′UTR), can influence gene expression if it is translated as well as transcribed, one can also employ a particular leader sequence. Suitable leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as for example described by Joshi (1987).

### Vectors

The present invention includes use of vectors for manipulation or transfer of genetic constructs. By “chimeric vector” is meant a nucleic acid molecule, preferably a DNA molecule derived, for example, from a plasmid, bacteriophage, or plant virus, into which a nucleic acid sequence may be inserted or cloned. A vector preferably is double-stranded DNA and contains one or more unique restriction sites and may be capable of autonomous replication in a defined host cell including a target cell or tissue or a progenitor cell or tissue thereof, or capable of integration into the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene, a herbicide resistance gene or other gene that can be used for selection of suitable transformants. Examples of such genes are well known to those of skill in the art.

The nucleic acid construct of the invention can be introduced into a vector, such as a plasmid. Plasmid vectors typically include additional nucleic acid sequences that provide for easy selection, amplification, and transformation of the expression cassette in prokaryotic and eukaryotic cells, e.g., pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked.

To facilitate identification of transformants, the nucleic acid construct desirably comprises a selectable or screenable marker gene as, or in addition to, the foreign or exogenous polynucleotide. The actual choice of a marker is not crucial as long as it is functional (i.e., selective) in combination with the plant cells of choice. The marker gene and the foreign or exogenous polynucleotide of interest do not have to be linked, since co-transformation of unlinked genes as, for example, described in U.S. Pat. No. 4,399,216 is also an efficient process in plant transformation.

Examples of bacterial selectable markers are markers that confer antibiotic resistance such as ampicillin, erythromycin, chloramphenicol or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327, an acetyltransferase gene from Streptomyces viridochromogenes conferring resistance to the selective agent phosphinothricin as, for example, described in EP 275957, a gene encoding a 5-enolshikimate-3-phosphate synthase (EPSPS) conferring tolerance to N-phosphonomethylglycine as, for example, described by Hinchee et al. (1988), a bar gene conferring resistance against bialaphos as, for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS), which confers resistance to imidazolinone, sulfonylurea or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known, a β-galactosidase gene encoding an enzyme for which chromogenic substrates are known, an aequorin gene (Prasher et al., 1985), which may be employed in calcium-sensitive bioluminescence detection; a green fluorescent protein gene (Niedz et al., 1995) or derivatives thereof, a luciferase (luc) gene (Ow et al., 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

Preferably, the nucleic acid construct is stably incorporated into the genome of, for example, the plant. Accordingly, the nucleic acid comprises appropriate elements which allow the molecule to be incorporated into the genome, or the construct is placed in an appropriate vector which can be incorporated into a chromosome of a plant cell.

One embodiment of the present invention includes a recombinant vector, which includes at least one polynucleotide molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

The level of NAC transcription factor can be elevated by increasing the level of expression of a nucleotide sequence that codes for the protein in a plant cell. The level of expression of a gene may be elevated by increasing the copy number per cell, for example by introducing a synthetic genetic construct comprising the coding sequence and a promoter that is operably connected thereto and that is functional in the cell. A plurality of transformants may be selected and screened for those with a favourable level and/or specificity of transgene expression arising from influences of endogenous sequences in the vicinity of the transgene integration site. A favourable level and pattern of transgene expression is one which results in a substantial enhancement of tolerance to stress conditions. Alternatively, a population of mutagenized seed or a population of plants from a breeding program may be screened for individual lines with increased levels of production of a NAC transcription factor as defined herein

### Recombinant Cells

Another embodiment of the present invention includes a recombinant cell comprising a host cell transformed with one or more recombinant molecules as defined herein, or progeny cells thereof. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules can remain extrachromosomal or can integrate into one or more sites within a chromosome of the transformed (i.e., recombinant) cell in such a manner that their ability to be expressed is retained. Preferred host cells are plant cells, more preferably cells of a wheat cell.

### Transgenic Plants

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, cotyledons, and seed coat), plant tissue (for example, vascular tissue, ground tissue, and the like), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, such as calli. Exemplary plant tissues in or from seeds are cotyledon, embryo and embryo axis. The invention accordingly includes plants and plant parts and products comprising these.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. A preferred species of hexaploid wheat is T. aestivum ssp aestivum (also termed “breadwheat”). Tetraploid wheat includes T. durum (also referred to herein as durum wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. Particularly preferred progenitors are those of the A genome, even more preferably the A genome progenitor is T. monococcum. A wheat cultivar for use in the present invention may belong to, but is not limited to, any of the above-listed species. Also encompassed are plants that are produced by conventional techniques using Triticum sp. as a parent in a sexual cross with a non-Triticum species (such as rye [Secale cereale]), including but not limited to Triticale.

The terms “seed” and “grain” are used interchangeably herein. “Grain” generally refers to mature, harvested grain but can also refer to grain after imbibition or germination, according to the context. Mature grain commonly has a moisture content of less than about 18-20%. “Developing seed” as used herein refers to seed at an immature stage as typically found in the plant from about 7 days post anthesis to a fully filled stage prior to maturity.

A “transgenic wheat plant” as used herein refers to a wheat plant that contains a nucleic acid construct not found in a wild-type plant of the same species, variety or cultivar. That is, transgenic plants (transformed plants) contain genetic material (a transgene) that they did not contain prior to the transformation. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. The genetic material is preferably stably integrated into the genome of the plant. The introduced genetic material may comprise sequences that naturally occur in the same species but in a rearranged order or in a different arrangement of elements, for example an antisense sequence. Plants containing such sequences are included herein in “transgenic plants”. A “non-transgenic plant” is one which has not been genetically modified by the introduction of genetic material by recombinant DNA techniques. In a preferred embodiment, the transgenic plants are homozygous for each and every gene that has been introduced (transgene) so that their progeny do not segregate for the desired phenotype.

As used herein, the term “compared to an isogenic plant” or similar phrases refer to a plant which is isogenic relative to the transgenic plant but without the genetic modification of interest. Preferably, the corresponding non-transgenic plant is of the same cultivar or variety as the progenitor of the transgenic plant of interest, or a sibling plant line which lacks the construct, often termed a “segregant”, or a plant of the same cultivar or variety transformed with an “empty vector” construct, and may be a non-transgenic plant. “Wild type”, as used herein, refers to a cell, tissue or plant that has not been modified according to the invention. Wild-type cells, tissue or plants may be used as controls to compare levels of expression of an exogenous nucleic acid or the extent and nature of trait modification with cells, tissue or plants modified as described herein. When determining whether a transgenic plant has enhanced tolerance to stress conditions, it is preferred that the transgenic plant and isogenic plant are grown under the same conditions and analysed at the stage of development.

Transgenic plants, as defined in the context of the present invention include progeny of the plants which have been genetically modified using recombinant techniques, wherein the progeny comprise the transgene of interest. Such progeny may be obtained by self-fertilisation of the primary transgenic plant or by crossing such plants with another plant of the same species. This would generally be to modulate the production of at least one protein defined herein in the desired plant or plant organ. Transgenic plant parts include all parts and cells of said plants comprising the transgene such as, for example, cultured tissues, callus and protoplasts.

The term progeny is used to refer to direct progeny of a plant produced by a method of the invention such as inserting an exogenous polynucleotide and generating a plant or crossing two plants one of which has been genetically modified as described herein, as well as indirect progeny such as second, third, fourth etc generation progeny.

Transgenic plants, as defined in the context of the present invention include plants (as well as parts and cells of said plants) and their progeny which have been genetically modified using recombinant techniques to cause production of at least one polypeptide defined herein in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

In a preferred embodiment, the transgenic plants are homozygous for each and every gene that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

As used herein, the “other genetic markers” may be any molecules which are linked to a desired trait of a wheat plant. Such markers are well known to those skilled in the art and include molecular markers linked to genes determining traits such disease resistance, yield, plant morphology, grain quality, other dormancy traits such as grain colour, gibberellic acid content in the seed, plant height, flour colour and the like. Examples of such genes are stem-rust resistance genes Sr2 or Sr38, the stripe rust resistance genes Yr10 or Yr17, the nematode resistance genes such as Cre1 and Cre3, alleles at glutenin loci that determine dough strength such as Ax, Bx, Dx, Ay, By and Dy alleles, the Rht genes that determine a semi-dwarf growth habit and therefore lodging resistance (Eagles et al., 2001; Langridge et al., 2001; Sharp et al., 2001). With specific regard to grain dormancy, other markers include the R gene for red grain colour (Himi et al., 2002), as well as markers described by Mares et al. (2005), Li et al. (2004), Kato et al. (2001), Mori et al. (2005) and Prada et al. (2004). Other genetic markers include genes for modification of starch metabolism such as, for example, a transgene encoding an RNA molecule that inhibits expression of the gene encoding GWD as described in WO2009/067551 or SBEIIa as described in WO2005/001098.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. No. 4,945,050 and U.S. Pat. No. 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. A particle delivery system suitable for use with the present invention is the helium acceleration PDS-1000/He gun is available from Bio-Rad Laboratories. For the bombardment, immature embryos or derived target cells such as scutella or calli from immature embryos may be arranged on solid culture medium.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. No. 5,451,513, U.S. Pat. No. 5,545,818, U.S. Pat. No. 5,877,402, U.S. Pat. No. 5,932,479, and WO 99/05265.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. No. 5,177,010, U.S. Pat. No. 5,104,310, U.S. Pat. No. 5,004,863, U.S. Pat. No. 5,159,135). Further, the integration of the T-DNA is a relatively precise process resulting in few rearrangements. The region of DNA to be transferred is defined by the border sequences, and intervening DNA is usually inserted into the plant genome.

Agrobacterium transformation vectors are capable of replication in E. coli as well as Agrobacterium, allowing for convenient manipulations as described (Klee et al., Plant DNA Infectious Agents, Hohn and Schell, (editors), Springer-Verlag, New York, (1985): 179-203). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different transgenic plants can also be mated to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, Breeding Methods for Cultivar Development, J. Wilcox (editor) American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011); Brassica (U.S. Pat. No. 5,463,174); peanut (Cheng et al., 1996); and pea (Grant et al., 1995).

Methods for transformation of wheat for introducing genetic variation into the plant by introduction of an exogenous nucleic acid and for regeneration of plants from protoplasts or immature plant embryos are well known in the art, see for example, CA 2,092,588, AU 61781/94, AU 667939, U.S. Pat. No. 6,100,447, WO 97/048814, U.S. Pat. No. 5,589,617, U.S. Pat. No. 6,541,257, and other methods are set out in WO 99/14314. Preferably, transgenic wheat plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts. The regenerable wheat cells are preferably from the scutellum of immature embryos, mature embryos, callus derived from these, or the meristematic tissue.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Tilling

Plants of the invention can be produced using the process known as TILLING (Targeting Induced Local Lesions IN Genomes) for detection of mutations in genes other than the exogenous polynucleotide. In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique.

TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### EXAMPLES

### Example 1

### Materials and Methods

### Plant Materials and General Growth Conditions

Spring bread wheat (Triticum aestivum L. cv. Bobwhite) plants were generally grown in a controlled-environment growth room in 1.5-litre pots containing University of California mix under night/day conditions of 16/20° C., 80/60% relative humidity and 16-h light with a photosynthetically active radiation flux of 500 μmol m⁻² s⁻¹ at the plant canopy level as described previously (Stephenson et al., 2010) except in some specific experiments as indicated below.

### Generation of Transgenic Wheat Lines

TaNAC69-1 expression constructs (HyDhn8s:TaNAC69-1:rice rbcS 3′ and HyDhn4s:TaNAC69-1:rice rbcS 3′) were made by inserting the coding region of TaNAC69-1 between the barley promoter (HvDhn8s or HvDhn4s) and rice rbcS 3′ in pSP72 vector using the construction method as described by Xiao and Xue (2001). The resulting expression constructs were used for production of TaNAC69 over-expression transgenic wheat lines. The correctness of the constructs was confirmed by nucleotide sequencing.

Immature embryos from Bobwhite SH 98 26 were used for wheat transformation using the particle bombardment method of Pellegrineschi et al. (2002). TaNAC69 expression cassettes and selectable marker gene cassettes [(rice act1: bar: rice rbcS3′, constructed from pAAI1GUSR and pBBar (Patel et al., 2000) and 35S-driven nptII from pCMneoSTLS2 (Maas et al., 1997)] were amplified from expression plasmids using PCR. The PCR-amplified gene cassettes were purified using a Qiagen column. Each TaNAC69 expression cassette was co-introduced with a selectable marker gene cassette into the immature embryos. The herbicide phosphinothricin or the antibiotic geneticin was used for selection of transformed calli. Herbicide- or geneticin-resistant plantlets were grown in a controlled environment growth room as described above.

The presence of a TaNAC69 transgene in the transgenic lines under study was verified by genomic real-time PCR using the primers corresponding to the rice rbcS 3′ region (sense primer: 5′-GCGAGGAGTCTGGTGGCAACT (SEQ ID NO: 30), antisense primer: 5′-AAGCAGAGCACGGCCGGTAA (SEQ ID NO:31). The genomic DNA was prepared from 15-30 mg of young leaf samples extracted with 0.6 ml of 1% (w/v) SDS and 0.5 M NaCl at 70° C. for 30 min after freeze-and-thaw of the leaf samples three times. The treated leaf samples were shaken vigorously for several hours to aid the release of genomic DNA into the solution. The genomic DNA was precipitated by adding an equal volume of iso-propanol, washed twice with 75% ethanol and dissolved in 50 μl of 2 mM Tris-HCl (pH 8) and 0.2 mM EDTA and 0.02 mg ml⁻¹ of DNase-free RNase A. After incubation at 37° C. for 3 h, 2 μl of the DNA solution was used in a 10-μl real-time PCR reaction for detection of the presence of TaNAC69 transgene with an ABI Prism 7900 sequence detection system (Applied Biosystems) using SYBR Green PCR Master Mix (Applied Biosystems) according to the manufacturer's instructions. Genomic DNA prepared from non-transgenic Bobwhite was used as a negative control. Transgenic lines with the presence of a TaNAC69 transgene in all 30 seedlings from a T1 line examined by the genomic real-time PCR were considered as apparently homozygous lines for phenotypic analyses.

### Drought and Salt Treatments Gene Expression Analysis

Drought stress of wheat plants was achieved by water deprivation of six-week-old plants grown in 1.5-litre pots in a controlled-environment growth room as described by Xue et al. (2006). Relative leaf water content was measured as described by Xue and Loveridge (2004). Leaf samples of plants with a relative leaf water content of 75±2% were used as drought-stressed samples for comparative gene expression analysis with non-stressed plants. For salt treatment, seeds were germinated in wet tissue at room temperature. On the fifth day after imbibition seedlings were transferred to a 250 mM NaCl solution containing 0.3 g L⁻¹ Hortico Aquasol fertilizer (Yates, Australia) and grown for 3 days in a growth cabinet (MLR-350; Sanyo, Osaka, Japan) at 20° C. and 16-h light with a photosynthetically active photon flux of 200 μmol m⁻² s⁻¹. Roots and shoots of seedlings (three seedlings were pooled as one sample) were harvested for gene expression analysis.

### Quantitative RT-PCR Analysis

Total RNA was isolated from plant material using Plant RNA reagent (Invitrogen, USA) according to the manufacturer's instructions. cDNA was synthesised using an oligo (dT₂₀) primer from total RNA samples that were pre-treated with RNase-free DNase I (Xue and Loveridge, 2004) and purified through a RNeasy column (Qiagen, Australia). Transcript levels were quantified with real-time PCR using an ABI Prism 7900 sequence detection system (Applied Biosystems) with a SYBR Green PCR Master Mix (Applied Biosystems) according to the manufacturer's instructions. The sequences of primer pairs used for real-time PCR are listed in Table 2. Genes encoding TaRPII36 (T aestivum RNA polymerase II 36 kDa subunit) and TaRP15 (T aestivum RNA polymerase I, II and III, 15 kDa subunit) were used as internal reference genes for calculating relative transcript levels of genes of the interest in each comparative analysis (Xue et al., 2008a; Stephenson et al., 2007). The relative expression levels were calculated according to Pfaffl (2001), as detailed in the study of Shaw et al. (2009). Statistical significance of differences between treatments or genotypes (transgenic and non-transgenic) was analysed using Student's t-test.

TABLE 2
Real-time PCR primers for TaNAC69-1 and TaNAC69-3 and TaNAC69-coregulated genes.

| | WHEAT | | | |
|---|---|---|---|---|
| Affymetrix | TC or | | Forward primer | Reverse primer |
| probe set ID | GenBank # | DESCRIPTION | (5'-3') | (5'-3') |
| Ta.14995.1.S1_x_at | TC452441 | Xyloglucan | CGTTCGTGGCGTCGTACA | GCGTCCAGCTCCTGGTACAT |
| | | endo- | (SEQ ID NO: 32) | (SEQ ID NO: 33) |
| | | transglycosylase | | |
| Ta.5339.1.S1_at | TC386834 | Dioxygenase | GTTGGTGAAGACGCTGTGCTAC | TGCTTTCTAGGGACTTGGAAGTGT |
| | | | (SEQ ID NO: 34) | (SEQ ID NO: 35) |
| Ta.24336.1.S1_x_at | TC399574 | HPPDase | CCCTTGAAGCCAAGCAATCT | GCTTCTCCGGCTCCAATTC |
| | | | (SEQ ID NO: 36) | (SEQ ID NO: 37) |
| Ta.8571.1.S1_x_at | TC383813 | Glyoxalase Ia | GAGAGCTTACCGOCGAAGAAG | GCTCTCGCACTGGAAGGAGAT |
| | | | (SEQ ID NO: 38) | (SEQ ID NO: 39) |
| Ta.8571.2.S1_x_at | TC396348 | Glyoxalase Ib | GTGGACCAGATCTTCTTCCATGA | GACGACCGGGAGCTTGTC |
| | | | (SEQ ID NO: 40) | (SEQ ID NO: 41) |
| Ta.21342.1.S1_x_at | TC378916 | Chitinase | CGACAACCTGGACTGCTACAAC | TCTCGCATCATATAGCCGATTG |
| | | 3-like | (SEQ ID NO: 42) | (SEQ ID NO: 43) |
| Ta.27265.1.S1_x_at | C1596583 | Xanthine | GGATAAGGATGGCGTGTGTTG | CGCTGCATGTATCGGCTAATC |
| | | dehydrogenase | (SEQ ID NO: 44) | (SEQ ID NO: 45) |
| Ta.14887.1.S1_at | TC398553 | beta-alanine | GAACTTATGCCGCCAGATCAA | GCATACATGTCATACCGAGCAGTC |
| | | synthase | (SEQ ID NO: 46) | (SEQ ID NO: 47) |
| Ta.14172.1.S1_at | TC447077 | ZIM family | CAATCACTGCCTCACTGCTTCT | GCCAATTAACGGGCGTAAGATA |
| | | protein | (SEQ ID NO: 48) | (SEQ ID NO: 49) |
| TaAffx.128418. | TC447464/ | Chitinase 3 | TGTGACCTCCTCGGTGTCAG | TCTTCTCGTATCCTATAGCCGATTG |
| 43.S1_at | TC447749 | | (SEQ ID NO: 50) | (SEQ ID NO: 51) |
| Ta.21267.1.S1_s_at | CJ949024 | Wali3-like | TACTGATCGCTCATGCTCAGAAC | GTTGTTGGGCGTGGAACAC |
| | | | (SEQ ID NO: 52) | (SEQ ID NO: 53) |
| Ta.4800.1.S1_at | TC446724 | Saccharopine | GAGGAACCTCCGTCCAAGAAAT | AAGACCGGCATCCAGGTTACTA |
| | | dehydrogenase- | (SEQ ID NO: 54) | (SEQ ID NO: 55) |
| | | like | | |
| Ta.4084.1.S1_at | TC385557 | Cellulose | AGCCCTTCGACTCGGGATTA | ATCGGCGTTCTTGACATCTTCT |
| | | synthase- | (SEQ ID NO: 56) | (SEQ ID NO: 57) |
| | | like | | |
| | | protein CslE | | |
| | AY625682 | TaNAC69-1 | TGCCTCCCGAAAACCCA | TTGTTCACCTTAGCCGTTGTTGT |
| | | | (SEQ ID NO: 58) | (SEQ ID NO: 59) |
| | DQ022843 | TaNAC69-3 | AACAATGGCTACGTGAACATCGA | AAACTGCCGCTGGACCTCTT |
| | | | (SEQ ID NO: 60) | (SEQ ID NO: 61) |

### Analysis of Polyethylene Glycol Induced Dehydration Tolerance

For polyethylene glycol (PEG) induced dehydration treatment, seeds were germinated in wet tissue at room temperature. On the fourth day after imbibition, seedlings with a similar shoot length were transferred to a solution containing 10% (w/v) PEG (MW=7000-9000) and 0.3 g L⁻¹ Hortico Aquasol fertilizer and grown for 14 days in a growth cabinet (MLR-350: Sanyo) as above. PEG concentrations were gradually increased to 16% on the fourth day of the treatment through water evaporation from the seedlings and directly from the solution and were maintained at this level for five more days by adding water or 0.3 g L⁻¹ Hortico Aquasol solution to compensate the water loss from evaporation. In the final five days of the treatment, the PEG concentrations were maintained at 18%. The plant length, root length and biomass of seedlings were measured after 2-week treatment.

### Measurement of Shoot Biomass Production Under a Combination of Mild Salt Stress and Water-Limited Conditions

For comparative shoot biomass production analysis, plants (transgenic and non-transgenic plants) were planted in the same bucket (10-L) containing 10 kg of soil (University of California mix), 3 L of 100 mM NaCl/6.6 mM CaCl₂/Aquasol (0.3 g L⁻¹) and grown at 14° C./18° C. (night/day) and 16-h light period in a controlled environmental room. Soil in the bucket was covered with a plastic sheet to reduce direct water evaporation from the soil. Additional 1 L of 200 mM NaCl/6.6 mM CaCl₂/Aquasol (0.3 g L⁻¹) was added to the bucket at the early sign of plants experiencing water deficit stress (at the early leaf wilting stage) during the growth period, and plants were allowed to grow further. Shoots were harvested and dried at 60° C. for 5 days for measurement of biomass production after plants showed severe drought stress (35 days after planting).

### Analysis of Water Use Efficiency

Seeds were germinated on wet tissue at room temperature. Five-day-old seedlings of similar shoot length were planted in plastic bottles (bottle size: 15-cm height and 7-cm diameter) with one plant per bottle. Each bottle contained 500 g of University of California Mix containing 14% of water and 120 ml of 0.3 g L⁻¹ of Aquasol fertilizer solution. The bottle was then covered with a plastic sheet with a 1-cm diameter hole and a 2-cm broken line at the centre. Plants were grown in a controlled environment growth room as described above for at least one month until all plant-extractable water in the bottle was used up. The total weight loss (i.e. water loss) from each bottle during the growth period was recorded. The shoot was harvested and dried at 60° C. for 5 days to record dry biomass weight. Water use efficiency is expressed as g of dry shoot biomass per 100 ml of water used.

### Analysis of Grain Yield and Grain Weight Under Water-Limited Conditions

Comparative analysis of grain yield per plant and grain weight between the transgenic wheat plants of the D4NAC69-L3 line (T3 plants) and non-transgenic control plants (cv. Bobwhite) under water-limited conditions was performed in a glass room. Plants were grown from mid July to early November (winter to spring) in big pots (30-cm diameter and 27-cm height, 6 plants per pot). A limited water supply regime started at the early booting stage and plants experienced dehydration stress during midday in sunny and hot days. Evaporative cooling was used to maintain growth temperatures, which ranged from 18° C. to 24° C. before the booting stage and from 18° C. to 28° C. afterwards due to the ineffectiveness of the evaporative cooler in hot days during the period of mid September to early November.

Analysis of Expression Data from Affymetrix Wheat Genome Array Datasets

Affymetrix wheat genome Array datasets were retrieved in CEL file format from http://www.ncbi.nlm.nih.gov/geo/ and http://www.ebi.ac.uk/microarray-as/aer/?#ae-main. The Affymetrix Wheat Genome Array contains 61,127 probe sets representing 55,052 transcripts for all 42 chromosomes in the wheat genome. Each set of raw Affymetrix data was normalized using robust multiarray average, a log scale measurement of expression developed by Irizarry et al. (2003), using the default settings for the Affymetrix package within Bioconductor, running within the R statistical programming environment (http://www.r-project.org), as described by Xue et al. (2008b). The normalised log values were converted to non-log values for expression correlation analysis. Pearson correlation analysis was used for the identification of genes that were co-regulated with TaNAC69 genes as described by Stephenson et al. (2010). Sequences representing probe sets significantly correlated with the expression levels of TaNAC69 were collected from the Triticum aestivum Gene Index (TaGI) database (Release 12.0, ftp://occams.dfci.harvard.edu/).

### DNA-Binding Activity Assays

pTaNAC69-CELD and pONAC131-CELD plasmids were constructed by translational fusion of the TaNAC69-1 or ONAC131 coding region sequence to the N-terminus of the 6×His-tagged CELD reporter enzyme (Xue, 2005). ONAC131 (Os12g03040.1) cDNA was obtained by PCR amplification of cDNA from the roots of Oryza sativa subspecies Japonica cv. Nipponbare using the following primers (sense: 5′-catggctagcCCGAGCAGCGGCGGCGCCA (SEQ ID NO: 62); antisense: 5′-cgggatccCTGCATCTGCAGATGATTGTTCAGCA (SEQ ID NO: 63)). The underlined sequences containing NheI and BamHI restriction sites used for cloning of the amplified cDNA directly into the NheI and BamHI restriction sites of the pTacLCELD6 x H is vector (Xue, 2005).

Synthetic biotin-labelled double-stranded oligonucleotide probes were synthesised by filling in partially double-stranded oligonucleotides using Taq polymerase reaction as described by Xue et al. (2006). For DNA-binding assays, 6×His-tagged CELD-fused NAC proteins were prepared from E. coli carrying the pTaNAC69-CELD or pONAC131-CELD plasmid and purified using Ni-NTA magnetic agarose beads (Xue, 2005). The DNA-binding activity of CELD-fused NAC proteins was measured as described previously (Xue, 2002) using StreptaWell High Bind (streptavidin-coated 8-well strips from Roche, Penzberg, Germany) and binding/washing buffer (25 mM HEPES/KOH, pH 7.0, 50 mM KCl, 2 mM MgCl₂) containing 0.5 mM DTT, 0.15 μg μl⁻¹ shared herring sperm DNA, 0.3 mg ml⁻¹ bovine serum albumin, 10% glycerol and 0.025% Nonidet P-40. 50,000 fluorescent units h⁻¹ of the CELD activity of NAC-CELD fusion protein and 1 pmol of synthetic oligonucleotides were used for NAC protein binding to biotin-labelled probes immobilised in the wells of streptavidin-coated 8-well strips. The unbound NAC-CELD fusion proteins were removed by three times of washing with ice-cold binding/washing buffer. The cellulase activity of the CELD fusion proteins bound to immobilised biotinylated probes was assayed by incubation in 100 μl of the CELD substrate solution [1 mM methylumbelliferyl β-D-cellobioside in 50 mM Na-citrate buffer, pH 6.0] at 40° C. for 2.5 h. A biotin-labelled double-stranded oligonucleotide without a target binding site was used as a control to measure background activity in the DNA-binding assays.

### Electrophoretic Mobility Shift Assays (EMSA)

EMSA assays were as described previously (Xue, 2005). Double-stranded synthetic oligonucleotides were labelled with digoxigenin at the 3′-end as described by Xue et al. (2006). TaNAC69-CELD protein was prepared and purified as above. Digoxigenin-labelled oligonucleotides (30 fmol) were incubated with purified TaNAC69-CELD protein (30 ng) in 15 μl of binding buffer (25 mM HEPES/KOH, pH 7.0, 50 mM KCl, 0.5 mM DTT, 2 mM MgCl₂, 0.2 μg μl⁻¹ poly d(AC-TG), 0.3 mg ml⁻¹ bovine serum albumin and 10% glycerol). After incubation at room temperature for 30 min, TaNAC69-CELD/DNA complexes were separated from free probes on a 6% polyacrylamide gel in a 40-mM Tris-acetate buffer (pH 7.5) containing 5 mM Na-acetate, 0.5 mM EDTA and 5% glycerol. TaNAC69-CELD/DNA complexes and free probes in the gels after electrophoresis were transferred to a Hybond N+ membrane. Alkaline phosphatase-conjugated anti-digoxigenin antibody and a chemiluminescent substrate, CDP-Star (Roche Diagnostics), were used for detection of digoxigenin according to the manufacturer's instructions.

### Example 2

### TaNAC69 Expression is Up-Regulated by Multiple Stresses

Four highly homologous wheat NAC genes (TaNAC69-1, TaNAC69-2, TaNAC69-3 and TaNAC69-4), which share amino acid identity of greater than 95% over the full-length protein sequences had been previously reported (Xue et al., 2006). These TaNAC69 genes were predominantly expressed in the root under normal conditions, but were markedly up-regulated by drought in both the leaf and root, as well as by cold in the leaf (Xue et al., 2006). To examine whether TaNAC69 expression was also responsive to other stress conditions, Affymetrix Wheat Genome Array datasets from http://www.ncbi.nlm.nih.gov/geo/ and http://www.ebi.ac.uk/microarray-as/aer/?#ae-main were retrieved and normalised as described in Example 1. An Affymetrix probe set (TaAffx.122104.1.S1_s_at, each probe set containing 11 probes) had a perfect or good sequence match with TaNAC69 genes (11 probe matches with TaNAC69-4, 10 probe matches with TaNAC69-3 and 6 probe matches with TaNAC69-1).

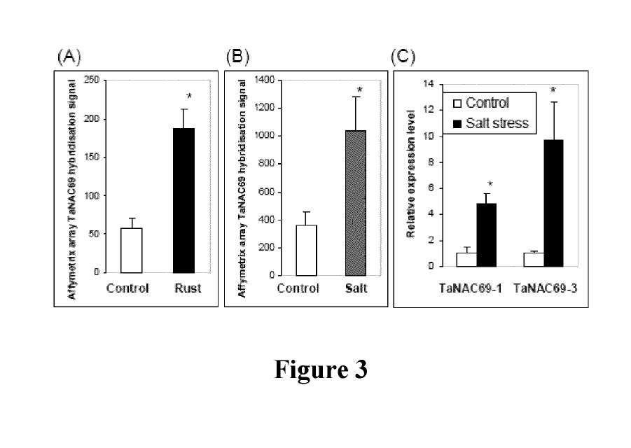

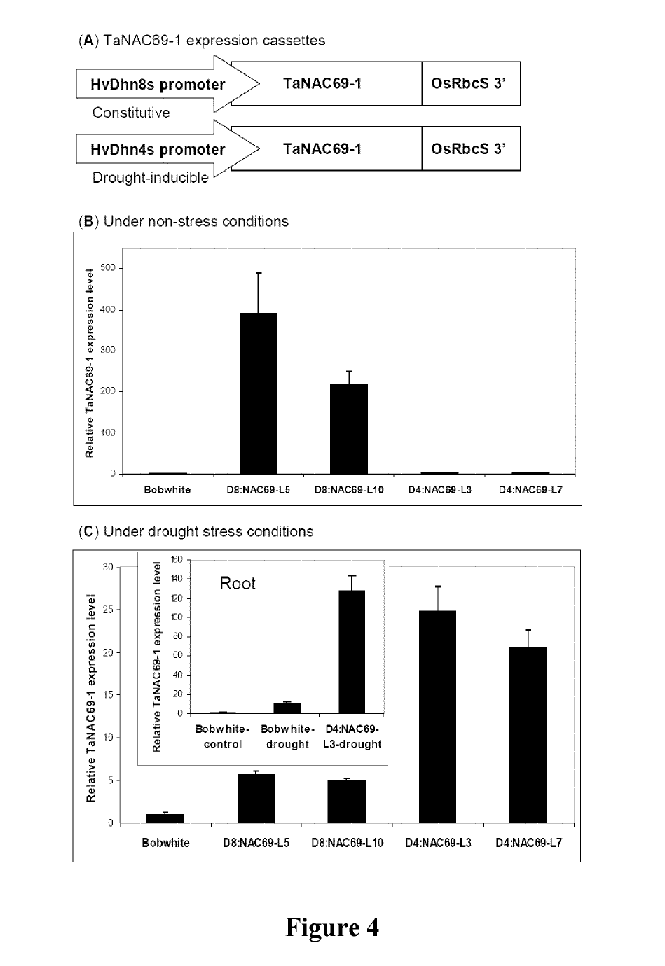

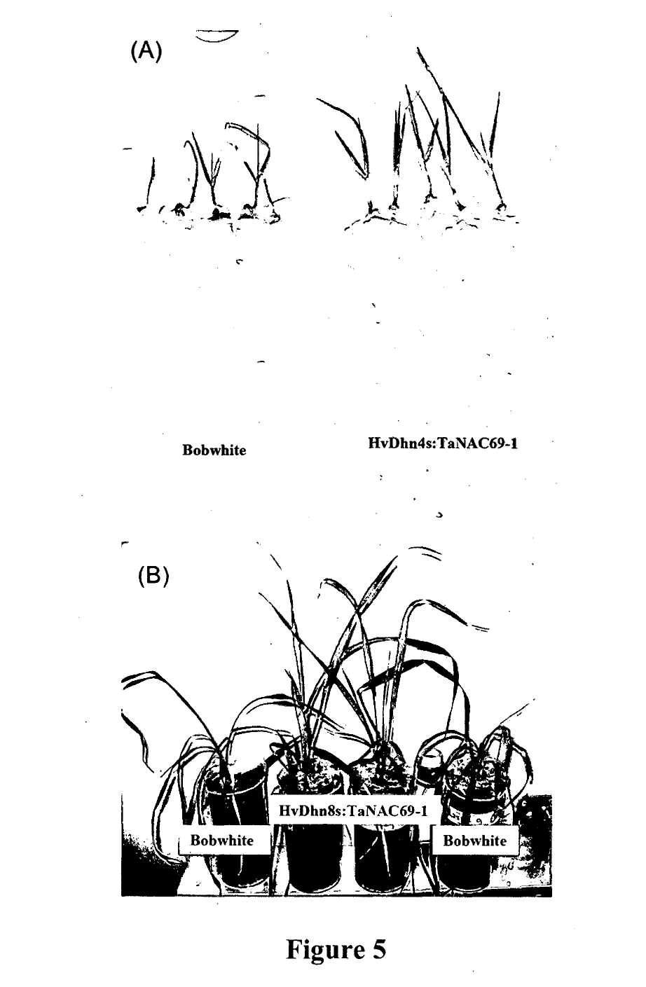

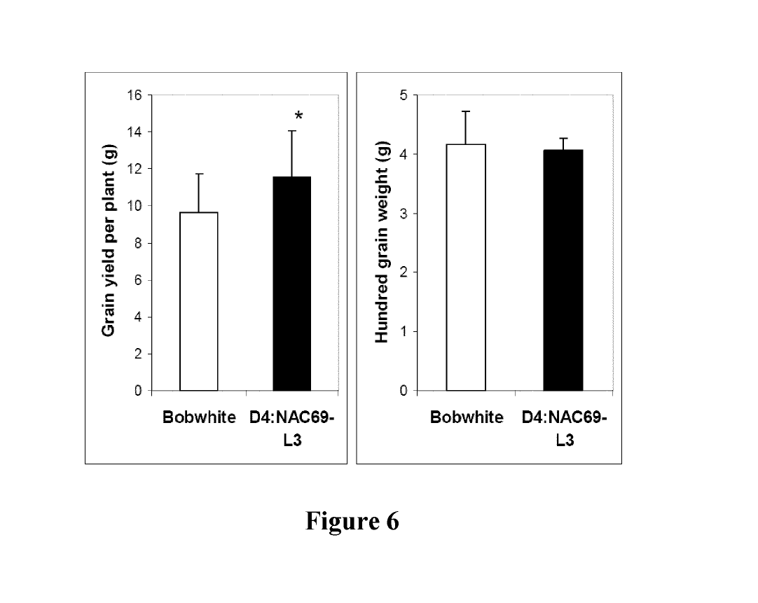

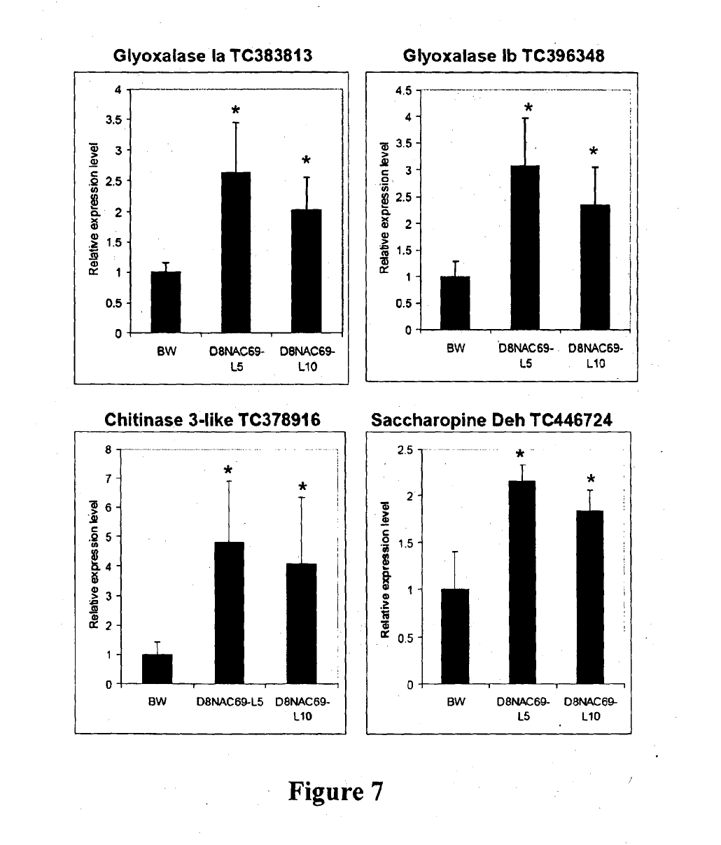

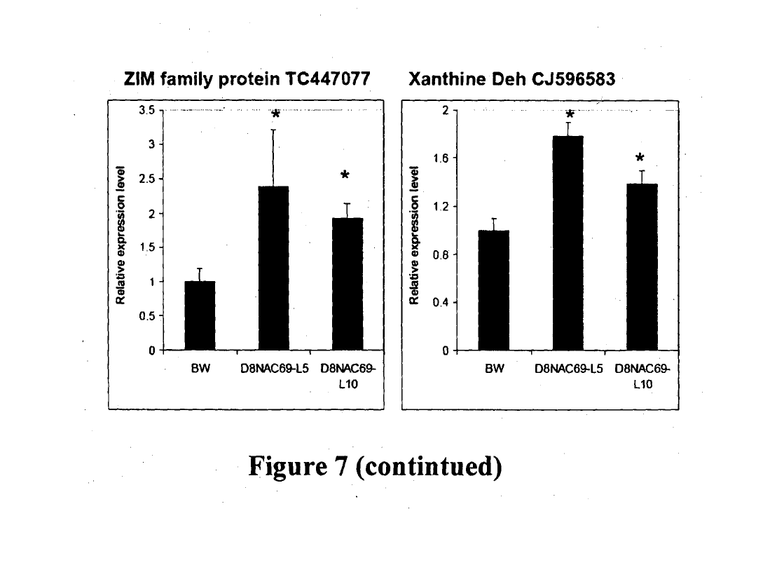

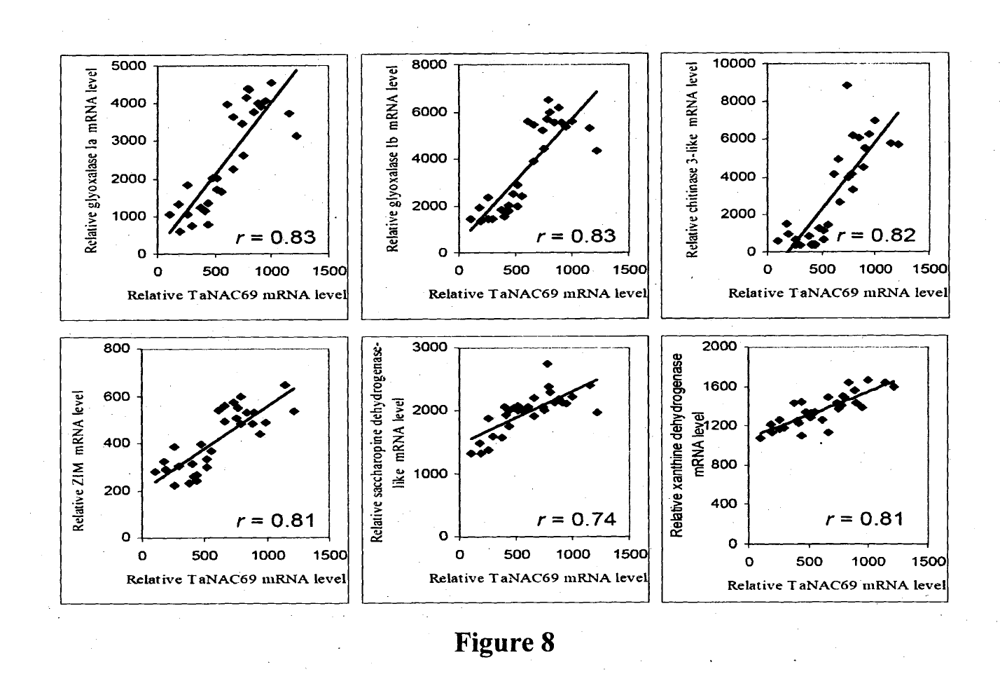

Analyses of the Affymetrix expression data from studies on wheat gene expression responses to salt stress (E-MEXP-971) and rust infection (GSE6227) revealed that TaNAC69 genes corresponding to the Affymetrix probe set TaAffx.122104.1.S1_s_at were also up-regulated in the roots of salt-stressed wheat plants and the leaves of a rust-resistant line of wheat after rust infection (FIGS. 3A and 1B). Quantitative RT-PCR analysis was performed as described in Example 1 to confirm the up-regulation by salt stress. As shown in FIG. 3C, the mRNA levels of TaNAC69-1 and TaNAC69-3 were up-regulated by 4-fold and 9-fold in salt-stressed seedling roots, respectively. TaNAC69 genes were also expressed in immature anthers of wheat and were markedly up-regulated by more than 3-fold in the mature anthers. A survey of Affymetrix Wheat Genome Array datasets generated from wheat stem/leaf sheath at anthesis (GSE9767, Xue et al., 2008b) and developing caryopses (E-MEXP-1193, Wan et al., 2008) revealed that there was no detectable hybridisation signal for TaNAC69 transcript (TaAffx.122104.1.S1_s_at). These expression analyses indicate that TaNAC69 genes were not constitutively expressed in all organs, including in stems and leaves, and were up-regulated under both abiotic and biotic stress conditions.

### Example 3

### Over-Expression of TaNAC69 in Transgenic Wheat Improves Dehydration Tolerance and Water Use Efficiency

In order to examine the biological role of TaNAC69 in wheat adaptation to drought stress, transgenic wheat lines carrying a TaNAC69-1 gene driven by either a constitutively expressed HvDhn8s or drought-inducible HvDhn4s promoter (FIG. 4A) were produced as described in Example 1. The promoters of these dehydrin (Dhn) genes were isolated from barley (Xiao and Xue, 2001). Expression levels of TaNAC69-1 in transgenic lines were determined by quantitative RT-PCR. A number of transgenic lines carrying HvDhn8s-driven TaNAC69 produced at least 100-fold more TaNAC69-1 mRNA in the leaves than the endogenous TaNAC69-1 level in the wild-type genotype (Bobwhite) under non-stressed conditions (FIG. 4B). In contrast, the HvDhn4s-driven TaNAC69-I lines produced very little TaNAC69-1 transcript under non-stressed conditions, but produced very high levels of this transcript under drought stress conditions (FIG. 4C), being >20 times higher than the drought up-regulated endogenous TaNAC69-1 mRNA level in the leaves of the parental genotype, Bobwhite. Enhanced expression (a 13-fold increase) of TaNAC69-1 was also observed in the roots of HvDhn4s:TaNAC69-I lines under drought stress in comparison with drought stressed Bobwhite roots (FIG. 4C insert). These expression data showed that the TaNAC69-1 transgenes were over-expressed in these transgenic lines.

To examine whether the over-expression of TaNAC69 had an impact on dehydration tolerance, seedlings of transgenic lines carrying HvDhn4s-driven or HvDhn8s-driven TaNAC69-1 constructs were treated with PEG-induced dehydration as described in Example 1, along with Bobwhite seedlings as the non-transgenic control. HvDnh4s-driven TaNAC69-1 transgenic wheat lines (D4:NAC69-L3 and D4:NAC69-L7) that had a very high level of TaNAC69-1 transcript under stress conditions showed enhanced tolerance to polyethylene glycol (PEG)-induced dehydration, compared to Bobwhite (Table 3 and FIG. 5A). These transgenic lines produced twice the amount of shoot and root biomass as that of Bobwhite after two-week PEG treatment of 5-day-old seedlings (Table 3). HvDhn4s:TaNAC69-1 lines had much longer root length than Bobwhite control plants under the dehydration stress (FIG. 5A). There were no obvious differences in dehydration tolerance under PEG treatment, as measured by biomass, between the HvDhn8s-driven TaNAC69-1 lines and Bobwhite. In addition, the relative biomass production was analysed for two representative transgenic lines grown in soil under a combination of mild salt stress and water-limitation, as described in Example 1. In a similar fashion to the biomass and root length experiments, HvDhn4s:TaNAC69-1 transgenic plants (D4:NAC69-L3, the high expressing line) produced significantly more shoot biomass under these stress conditions at the early vegetative stage. No significant difference in plant biomass was observed between HvDhn8s:TaNAC69-1 plants (D8:NAC69-L5) and Bobwhite plants (Table 4).

TABLE 3
Shoot and root growth of HvDhn4s:TaNAC69-1 transgenic lines
(D4:NAC69-L3 and D4:NAC69-L7) under PEG-induced
dehydration stress in comparison with non-transgenic Bobwhite.

| | Bobwhite | D4:NAC69-L3 | D4:NAC69-L7 |
|---|---|---|---|
| Green shoot length (cm) | 5.8 ± 1.9 | 10.3 ± 0.9* | 9.9 ± 0.7* |
| Root length (cm) | 10.4 ± 2.9 | 17.7 ± 1.6* | 16.8 ± 1.2* |
| Fresh shoot wt (mg) | 74.0 ± 31.5 | 149.0 ± 11.1* | 144.6 ± 9.1* |
| Dry shoot wt (mg) | 23.0 ± 9.4 | 42.4 ± 3.1* | 41.6 ± 3.8* |
| Fresh root wt (mg) | 57.8 ± 23.7 | 144.3 ± 19.5* | 131.0 ± 15.4* |
| Dry root wt (mg) | 12.0 ± 4.9 | 28.8 ± 4.2* | 26.8 ± 3.3* |

Values are means ± SD of 5 plants.
Statistical significance of the differences between each transgenic line (at the T2 stage) and Bobwhite control was determined using Student's t-test.
*P < 0.01.
wt, weight.

TABLE 4
Biomass production of transgenic lines over-expressing TaNAC69
under a combination of mild salt and water-limited
conditions in comparison with non-transgenic Bobwhite.

| | Experiment 1 | Experiment 2 |
|---|---|---|
| | Dry biomass/ | Dry biomass/ |
| Genotype | plant (g) | plant (g) |
| D8:NAC69-L5 | | 4.59 ± 0.65 |
| D4:NAC69-L3 | 5.14 ± 0.87 | |
| Bobwhite control | 3.75 ± 0.78 | 4.10 ± 0.81 |
| P-value | 0.016 | 0.27 |

Values are means ± SD of 6 plants.
Statistical significance of the differences between each transgenic line and Bobwhite control was determined using Student's t-test.

Transgenic lines over-expressing TaNAC69 were also analysed for water use efficiency at the early vegetative stage in a controlled environmental room with a restricted amount of water as described in Example 1. In this use water efficiency test, each plant was grown in a bottle containing the same amount of soil and water. Plants were allowed to grow until all of the available water in each bottle was used up i.e. no water addition after planting. Plants experienced water deficit when the soil water content was reduced mainly by plant uptake, as evaporation directly from soil was minimised by plastic sheeting covering the soil surface (see FIG. 5B). HvDhn8s:TaNAC69-1 (D8:NAC69) transgenic plants appeared to grow better than the Bobwhite controls for the same amount of water (FIG. 5B). All three D8:NAC69 transgenic lines produced more biomass with a given amount of water at the early vegetative growth period than Bobwhite and non-transgenic segregants (null lines) (Table 5), suggesting that constitutive over-expression of TaNAC69 confers high water use efficiency. TaNAC69-1 driven by a drought-inducible promoter (HvDhn4s) also produced more biomass per unit of water than Bobwhite, but the difference was relatively small, with the water use efficiency being only ˜10% higher in HvDhn4sTaNAC69-1 lines than Bobwhite.

The other plant phenotypes of the TaNAC69 over-expressing plants appeared to be normal. The grain weight and yield (yield per plant) of HvDhn4s:TaNAC69-1 transgenic plants were similar to those of Bobwhite plants, grown in a controlled environmental room under well-watered conditions (Table 6). However, the grain weight of constitutively expressed TaNAC69-1 lines (HvDhn8s:TaNAC69-1) was significantly reduced, although the maturity biomass production was not significantly affected (Table 6).

TABLE 5
Comparative analysis of water use efficiency (WUE)
between HvDhn8:TaNAC69-1 (D8:NAC69)
T2 transgenic and non-transgenic plants at the early
vegetative stage with a restricted amount of water.

| | WUE (g dry shoot | |
|---|---|---|
| | biomass 100 ml⁻¹ water) | |
| Genotype | Mean | SD |
| Transgenic (T2 plants) | | |
| D8:NAC69-L3 | 0.577 | 0.032* |
| D8:NAC69-L5 | 0.618 | 0.026* |
| D8:NAC69-L10 | 0.607 | 0.039* |
| Transgenic mean | 0.601 | |
| Non-transgenic | | |
| Bobwhite | 0.526 | 0.039 |
| D8:NAC69-L3 Null | 0.513 | 0.026 |
| D8:NAC69-L10 Null | 0.505 | 0.026 |
| Non-transgenic mean | 0.515 | |

Values are mean ± SD of 8 plants.
Null lines are segregants containing no TaNAC69-1 transgene.
Statistical significance of the differences between each transgenic line and Bobwhite control was determined using Student's t-test.
*P > 0.01.

TABLE 6
The maturity biomass, spike number, grain weight and yield of T2
homozygous TaNAC69-1 transgenic lines in comparison with Bobwhite.
Plants were grown in a controlled environmental room under well-watered conditions.

| | Headless | | | |
|---|---|---|---|---|
| Transgenic | straw wt at | Spike | | |
| lines | maturity per | number | Hundred | Grain yield |
| or control | plant (g) | per plant | grain wt (g) | per plant (g) |
| Bobwhite | 14.45 ± 0.89 | 8.2 ± 0.7 | 5.15 ± 0.13 | 22.71 ± 1.78 |
| (control) | | | | |
| D4:NAC69-L3 | 15.57 ± 0.62* | 8.1 ± 0.5 | 5.10 ± 0.24 | 22.82 ± 2.03 |
| D4:NAC69-L7 | 14.18 ± 0.82 | 7.8 ± 0.5 | 5.12 ± 0.14 | 21.83 ± 1.84 |
| D8:NAC69-L5 | 15.14 ± 1.50 | 8.5 ± 0.6 | 4.73 ± 0.28* | 21.37 ± 1.52 |
| D8:NAC69-L10 | 13.65 ± 0.81 | 7.9 ± 0.4 | 4.92 ± 0.16* | 21.14 ± 1.15 |

Values are means of ± SD of six pots (4-L size, two plants per pot) for each line.
The homozygous transgenic lines were used for analysis.
Statistical significance of differences between transgenic line and Bobwhite was analysed by Student's t-test and indicated by *(P < 0.05).
Hundred grain wt is the weight (wt) per 100 grains.

To provide a preliminary assessment on the effect of TaNAC69 over-expression on the grain yield under water-limited conditions, a comparative analysis was performed of the grain yield and grain weight between the HvDhn4s:TaNAC69-1 line (D4:NAC69-L3 at the T3 stage) and Bobwhite under water-limited conditions. A limited water supply regime commenced at the early booting stage. As shown in FIG. 156, the grain yield per plant of the D4:NAC69-L3 line was significantly higher than that of Bobwhite, while there was no difference in grain weight between the D4:NAC69-L3 line and Bobwhite.

### Example 4

### TaNAC69 Over-Expression Enhances the mRNA Levels of Stress Up-Regulated Genes

To elucidate the target genes of TaNAC69, a genome-wide correlation analysis was performed between TaNAC69 expression levels and those of other genes using four sets of wheat Affymetrix Wheat Genome Array data. These were: (1) the root samples of control and salt-stressed plants from salt-tolerant and susceptible genotypes (E-MEXP-1193, Mott and Wang 2007), (2) the whole grain samples at 5 days after anthesis from 39 genotypes grown in the field in Canada (GSE4935, Jordan et al., 2007), (3) the flag leaf samples of wheat plants with and without rust infection in rust-tolerant and susceptible genotypes (GSE6227, Hulbert et al., 2007) and (4) anther samples at the various developmental stages of wheat (GSE6027, Crismani et al., 2006). These four Affymetrix datasets were selected for expression correlation analysis because the mRNA levels of TaNAC69 genes (TaNAC69-1, TaNAC69-3 and TaNAC69-4) varied considerably among samples (>2-fold between the highest and lowest levels) within each of these four datasets. Such variation is required for meaningful expression correlation analysis. Potential TaNAC69 target genes were selected if the transcript levels of these genes were significantly correlated with the TaNAC69 transcript levels in a minimum of three of the four separate datasets.

To improve the accuracy of regulator and target gene relationship predictions, positively correlated transcripts were selected that were also expressed higher than their potential regulator (TaNAC69), based on the general assumption of signal amplification from a transcription factor to its target genes. This global expression analysis identified a number of potential target genes (Table 7). All of these genes are up-regulated under abiotic and/or biotic stress conditions based on Affymetrix Wheat Genome Array data. Some related genes were known to have a role in detoxification (e.g., glyoxalase I family protein), antioxidant function (e.g., 4-hydroxyphenylpyruvate dioxygenase) or defence against pathogens such as chitinases. Among these TaNAC69 co-expressed genes, there was one ZIM family gene (Ta.14172.1.S1_at) which encoded a putative transcription factor.

TABLE 7
TaNAC69-coregulated genes, based on analysis of Affymetrix Wheat Genome Array datasets: (1) the root samples of control and salt-stressed
plants from salt-tolerant and susceptible genotypes (E-MEXP-1193, n = 30, Mott and Wang 2007), (2) the whole grain samples at 5 days
after anthesis from 39 genotypes grown in the field in Canada (GSE4935, n = 78, Jordan et al. 2007), (3) the flag leaf samples of
wheat plants with and without rust infection in rust-tolerant and susceptible genotypes (GSE6227, n = 36, Hulbert et al., 2007) and
(4) anther samples at the various developmental stages of wheat (GSE6027, n = 21, Crismani et al. 2006). All genes listed in this
table are upregulated by salt stress in the root and have a higher expression level than TaNAC69 in the root (Affymetrix dataset: E-MEXP-971).

| | | | Correlation coefficient between the mRNA levels | | | |
|---|---|---|---|---|---|---|
| | | | of TaNAC69 & its coregulated genes | | | |
| | Wheat TC or | | Salt & | Whole grain | Rust & | |
| Affymetrix probe set ID | GenBank # | Description | control root | 5DAA | control leaf | Anthers |
| Ta.14995.1.S1_x_at | TC452441 | Xyloglucan endo-transglycosylase | 0.91 | 0.96 | 0.79 | 0.57 |
| Ta.1022.2.S1_x_at | TC370488 | Rhodanese Homology Domain protein | 0.90 | 0.74 | 0.04 | 0.56 |
| Ta.12764.1.A1_at | TC444164 | R-hydratase-like hot dog fold of the 17-beta- | 0.89 | 0.81 | 0.93 | 0.50 |
| | | hydroxysteriod dehydrogenase (HSD), and | | | | |
| | | Hydratase-Dehydrogenase-Epimerase (HDE) | | | | |
| | | protein | | | | |
| Ta.28799.1.S1_a_at | TG392134 | Isopenicillin N synthase and related | 0.89 | 0.65 | 0.98 | 0.57 |
| | | dioxygenases | | | | |
| Ta.4035.3.S1_at | TC375124 | Beta-glucosidase | 0.89 | 0.62 | 0.86 | 0.46 |
| Ta.5339.1.S1_at | TC386834 | Dioxygenase family protein | 0.88 | 0.77 | 0.96 | 0.61 |
| Ta.7134.2.S1_at | TC374252 | electron transfer flavoprotein alpha | 0.88 | 0.68 | 0.91 | 0.64 |
| | | subunit | | | | |
| Ta.24336.1.S1_x_at | TC399574 | 4-hydroxyphenylpyruvate dioxygenase | 0.86 | 0.86 | 0.95 | −0.53 |
| | | (HPPDase) | | | | |
| Ta.24477.1.S1_at | TC401225 | Unknown function protein (similar to | 0.85 | 0.77 | 0.98 | 0.58 |
| | | Arabidopsis reversion-to-ethylene sensitivity1 | | | | |
| | | protein) | | | | |
| Ta.8571.1.S1_x_at | TC383813 | Glyoxalase I family protein | 0.83 | 0.75 | 0.98 | 0.53 |
| | | (lactoylglutathione lyase) | | | | |
| Ta.8571.2.S1_x_at | TC396348 | Glyoxalase I family protein | 0.83 | 0.79 | 0.98 | 0.55 |
| Ta.29349.1.S1_x_at | TC380786 | BRI1-KD interacting protein 128-like | 0.83 | 0.67 | 0.81 | 0.52 |
| Ta.21342.1.S1_x_at | TC378916 | chitinase 3-like (Class I chitinase) | 0.82 | 0.84 | 0.46 | 0.60 |
| Ta.27265.1.S1_x_at | CJ596583 | Xanthine dehydrogenase, molybdopterin | 0.81 | 0.79 | 0.96 | 0.53 |
| | | binding domain containing protein | | | | |
| Ta.14887.1.S1_at | TC398553 | Beta-alanine synthase | 0.81 | 0.72 | 0.89 | 0.35 |
| Ta.14172.1.S1_at | TC447077 | ZIM motif containing protein (CCT and Tify | 0.80 | 0.91 | −0.02 | 0.58 |
| | | domains) | | | | |
| TaAffx.128418.43.S1_at | TC447464 | Chitinase 3 (Class I chitinase) | 0.80 | 0.86 | 0.76 | 0.59 |
| Ta.21267.1.S1_s_at | CJ949024 | Aluminium-inducibie protein 3 (Wali3)-like | 0.80 | 0.93 | 0.95 | 0.09 |
| | | protein | | | | |
| Ta.4800.1.S1_at | TC446724 | Saccharopine dehydrogenase-like protein | 0.74 | 0.84 | 0.98 | 0.65 |
| Ta.4084.1.S1_at | TC385557 | Cellulose synthase-like protein CslE | 0.68 | 0.90 | 0.49 | 0.50 |

Fourteen of the top-listed candidate target genes shown in Table 3 were analysed for changes in expression level in the leaves of constitutive TaNAC69-1 over-expressing lines grown under non-stress conditions. Six of them (glyoxalase Ia, glyoxalase Ib, chitinase 3-like (class I chitinase), saccharopine dehydrogenase-like, ZIM family protein and xanthine dehydrogenase) were found to be significantly up-regulated in the leaves of transgenic lines constitutively over-expressing TaNAC69-1 grown under non-stress conditions (FIG. 7). As shown in FIG. 8, these six genes were highly correlated in expression with TaNAC69 genes in the Affymetrix E-MEXP-971 dataset, which is comprised of root samples from five wheat genotypes with or without salt treatment (Mott and Wang, 2007).

### the DNA-Binding Activity of TaNAC69-1 to Promoter Elements

DNA-binding sequences of TaNAC69-1 were previously characterised using an in vitro binding site selection system (Xue, 2005; Xue et al., 2006). TaNAC69-1 had two consensus DNA-binding sites with each consisting of two motifs spanning 23-24 bp. Therefore, it was thought to have highly stringent recognition of DNA-binding sequences. As the genomic sequence of wheat is still incomplete whereas the complete rice genome sequences was available; a search was made for TaNAC69-1 binding sites in the promoters of rice homologues of the six TaNAC69-1 up-regulated genes mentioned above. TaNAC69 binding site-like sequences were found in the promoters, i.e. within 1 kb upstream of the translation start codon; of three rice homologues of the six TaNAC69-1 up-regulated genes. These rice genes were genes encoding class I chitinase (Os06g0726100), glyoxalase I family (Os05g0171900) and ZIM family protein (Os03g0180900). In vitro DNA-binding analysis showed that TaNAC69-1 was capable of binding to these promoter elements (Table 8). In particular, TaNAC69-1 binding activity to the site I of the rice chitinase promoter was similar to those of the TaNAC69-1 preferred binding sequences. The 32-bp fragment of the site I element of the rice chitinase promoter was also present in the 5′-untranslated region of Arabidopsis glycoxalase I family gene (At1g80160) (Table 8).

TABLE 8
TaNAC69 and ONAC131 binding activities to the elements present in the promoters
of rice or Arabidopsis homologues of TaNAC69 up-regulated genes in comparison
with their binding activities to in vitro TaNAC69-selected binding sequences
(SO1 and SO39). The sequences of TaNAC69 binding motifs or similar to the consensus
binding motifs in each oligonucleotide are underlined. The consensus binding
motifs of TaNAC69 binding site I and II with high binding affinity are
rrwkmCGTrnnnnnyACGtmayy (SEQ ID NO: 23) and rswvktynnnnnnnnYACGwcwct (SEQ ID NO: 24)
(k = gt, m = ac, r = ag, s = cg, v = acg, w = at, y = et), respectively, where the
bases in upper-case letters are major determinants of its binding affinity
(Xue et al., 2006). SO1m14, SO1m28 and SO39m6 are base substitution mutants
of SO1 and SO39. Mutated bases are in lower-case letters. Values are
mean ± SD of three replicated assays.

| | | | Relative binding activity | |
|---|---|---|---|---|
| Name | Accession # | Oligonucleotide sequence | TaNAC69-1 | ONAC131 |
| TaNAC69 high affinity | | GAGATCCGTGCACAGTACGTAACTGTTACA | 1.00 ± 0.06 | 1.00 ± 0.03 |
| binding site I (SO1) | | (SEQ ID NO: 64) | | |
| SO1m14 | | GAGATCCGTGCACAGTAaaTAACTGTTACA | 0 | 0 |
| | | (SEQ ID NO: 65) | | |
| SO1m28 | | GAGATCtGTGCACAGTACGTAACTGTTACA | 0.01 ± 0.01 | 0.01 ± 0.01 |
| | | (SEQ ID NO: 66) | | |
| TaNAC69 high affinity | | GAGGTGTTTAATGTTTACACGTCTCTAGTG | 0.94 ± 0.05 | 0.93 ± 0.05 |
| binding site II (SO39) | | (SEQ ID NO: 67) | | |
| SO39m6 | | GAGGcccTTAATGTTTACACGTCTCTAGTG | 0.19 ± 0.02 | 0.18 ± 0.01 |
| | | (SEQ ID NO: 68) | | |
| Rice glyoxalase I | Os05g0171900 | CACTTGACACGTGGGATCCACGTCACCATCCT | 0.27 ± 0.03 | 0.29 ± 0.03 |
| family protein | | (SEQ ID NO: 69) | | |
| Rice chitinase site I | Os06g0726100 | GCCAGCGTTCTGTTTCTGTACGTCTCTCTGGG | 1.04 ± 0.06 | 1.07 ± 0.03 |
| | | (SEQ ID NO: 70) | | |
| Rice chitinase site II | Os06g0726100 | AATCCACCGCCATAAATCTACGTAACAAGTCA | 0 | 0 |
| | | (SEQ ID NO: 71) | | |
| Rice chitinase site III | Os06g0726100 | TTCCGTAGTTTACTGGATCACGACACACACAC | 0.14 ± 0.01 | 0.15 ± 0.02 |
| | | (SEQ ID NO: 72) | | |
| Rice ZIM family protein | Os03g0180900 | GATTTGTCTCGTAATTTACACGTAATCTGTGT | 0.46 ± 0.03 | 0.43 ± 0.05 |
| | | (SEQ ID NO: 73) | | |
| Arabidopsis | At1g80160.1 | GCCAGCGTTCTGTTTCTGTACGTCTCTCTGGG | 1.04 ± 0.06 | 1.07 ± 0.03 |
| glyoxalase I | | (SEQ ID NO: 74) | | |
| family protein | | | | |

TaNAC69-1 binding to the site present in the promoter of rice glyoxalase I family gene (Os05g0171900) was the weakest among the three rice genes tested, being 27% of the binding activity of the preferred TaNAC69-1 binding site (SO1). However, this percentage of the binding activity was still substantial, as it also complexed with TaNAC69-DNA as observed in electrophoretic mobility shift assays. In particular, unlike in many other binding assays which use tandem DNA repeats containing multiple binding sites as probes for enhancing transcription factor binding, oligonucleotides containing a single binding site were used for DNA-binding analysis in this study. Therefore, the detection of TaNAC69 binding to these sites in the rice promoters was likely to represent its true binding ability. The DNA-binding activity of one of the closest rice homologues of TaNAC69, ONAC131 (Os12g03040, Fang et al., 2008), to these sites was also tested. Similar binding results were observed (Table 8).

### Example 5

### Discussion

Expression data clearly showed that TaNAC69 transcription was up-regulated by multiple stress factors: drought, cold, high salt concentration and pathogens. The TaNAC69 genes were not expressed or expressed at a very low level in wheat stems, developing caryopses and leaves under non-stress conditions. However, TaNAC69 genes were constitutively expressed at a moderate level in the roots of wheat plants under non-stress conditions with >10-fold up-regulation during drought stress (Xue et al., 2006). Many dehydration up-regulated genes are also known to be expressed in the roots of cereal plants under non-stress conditions, such as barley HVA1 (Xue and Loveridge, 2004). These expression data imply that the biological function of TaNAC69 is associated with abiotic and biotic stress adaptation in wheat.

To elucidate the biological role of TaNAC69 in wheat drought adaptation, transgenic wheat lines over-expressing TaNAC69-1 were generated. Two constitutively expressed transgenic lines had the TaNAC69-1 mRNA level in the leaves under non-stress conditions >100 fold higher than plants of the wild-type, non-transgenic cultivar Bobwhite and also had significantly higher TaNAC69-1 expression under drought stress conditions compared to Bobwhite. These transgenic lines showed improved water use efficiency under a restricted water condition, but exhibited no significant difference from Bobwhite under constant stress conditions, such as the PEG-induced dehydration or a combination of mild salt stress and water limitation. In contrast, TaNAC69-1 transgenic lines driven by a strong drought-inducible promoter (HvDhn4s) showed significant improvement in tolerance to PEG-induced dehydration and under the mild salt stress/water-limited conditions and produced more shoot and root biomass than Bobwhite. Thus, these data provided experimental evidence on the role of drought up-regulated NAC transcription factors in drought adaptation in wheat.

The difference in dehydration tolerance under constant stress conditions between transgenic lines carrying HvDhn8s- and HvDhn4s-driven TaNAC69-1 constructs was likely to be related to the high expression level of HvDhn4s-driven TaNAC69-1 transgene in the roots and leaves of these transgenic lines under stress conditions. As the endogenous TaNAC69 transcript level was also markedly up-regulated in wheat leaves and roots during drought stress (Xue et al., 2006), as well as other drought up-regulated transcription factors in wheat such as TaNF-Y, TaZFP and TaD of family members (Stephenson et al., 2007; Kam et al., 2008; Shaw et al., 2009), it was likely that the transcript level of TaNAC69-1 transgene in HvDhn8s-driven transgenic lines was not sufficient for significant improvement of dehydration tolerance under constant stress conditions, but was sufficient only under temporary dehydration stress. The drought up-regulated Arabidopsis NAC transcription factors (ANAC019, ANAC055 and ANAC072) share high sequence similarity in the NAC domain with TaNAC69-1 (Xue et al., 2006) and have been shown to act as transcriptional activators (Tran et al., 2004; Fujita et al., 2004).

Expression correlation analysis revealed that the mRNA levels of a number of stress up-regulated genes were closely correlated with those of TaNAC69 genes (TaNAC69-1, TaNAC69-3 and TaNAC69-4) in several Affymetrix datasets. Quantitative RT-PCR analysis of 14 of these genes in TaNAC69 over-expressing lines identified six of them with significantly higher mRNA levels in the HvDHn8s:TaNAC69-1 transgenic lines than in plants of cultivar Bobwhite under non-stress conditions. Glyoxalase I converts a toxic intermediate metabolite methylglyoxal to S-D-lactoylglutathione and the methylglyoxal level can increase several folds in plants under salinity and drought conditions (Yadav et al., 2005). Xanthine dehydrogenase is a rate-limiting enzyme involved in purine degradation and remobilization (Brychkova et al., 2008). Class I chitinases are involved in plant responses to both biotic and abiotic stresses (Tateishi et al., 2001; Fossdal et al., 2007). However, the role of class I chitinase in drought adaptation is still not clear. The wheat chitinase 3-like gene is a class I chitinase and is highly homologous to rye chitinase 9 and Bromegrass chitinase 1, which have been shown to possess antifreeze property (Yeh et al., 2000; Nakamura et al., 2008). Saccharopine dehydrogenase is involved in lysine catabolism and its role in drought adaptation is not clear at the present. TaNAC69 up-regulated ZIM family gene is a putative transcription factor. As it is up-regulated during abiotic stress (Affymetrix dataset: E-MEXP-971), it is likely to be involved in the regulation of other stress genes in wheat. Based on these up-regulated genes, TaNAC69 is thought to confer better drought adaptation mainly by enhancing the levels of stress protection genes.

The promoters of three rice homologues [class I chitinase (Os06g0726100), ZIM family protein (Os03g0180900) and glyoxalase I family protein (Os05g0171900)] of TaNAC69 up-regulated genes were found to contain TaNAC69 binding sites. These DNA-binding data provided additional experimental evidence that the chitinase, ZIM family gene and glyoxalase I family gene were likely to be direct target genes of the TaNAC69 transcription factors.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from U.S. 61/468,235 filed 28 Mar. 2011, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abdullah et al. (1986) Biotechnology 4: 1087.
Aida et al. (1997) Plant Cell 9: 841-857.
Balazadeh et al. (2010) Plant J. 62: 250-264.
Bevan et al. (1983) Nucl. Acid Res. 11: 369-385.
Bhatnagar-Mathur et al. (2007) Plant Cell Rep. 26: 2071-2082.
Brychkova et al. (2008) Plant J. 54: 496-509.
Busk et al. (1997) The Plant Journal 11:1285-1295.
Capecchi (1980) Cell 22: 479-488
Cheng et al. (1996) Plant Cell Rep. 15:653-657.
Christensen & Quail (1989) Plant Molecular Biology 12:619-632.
Christensen et al. (1992) Plant Molecular Biology 18:675-689.
Clapp (1993) Clin. Perinatol. 20: 155-168.
Comai et al. (2004) Plant J 37: 778-786.
Crismani et al. (2006) BMC Genomics 7: 267.
Curiel et al. (1992) Hum. Gen. Ther. 3: 147-154.
Delessert et al. (2005) Plant J. 43: 745-757.
Duval et al. (2002) Plant Mol. Biol. 50: 237-248.
Eagles et al. (2001) Australian Journal of Agricultural Research 52: 1349-1356
Eglitis et al. (1988) Biotechniques 6: 608-614.
Ernst et al. (2004) EMBO Rep. 5: 297-303.
Fang et al. (2008) Mol. Genet. Genomics 280: 547-563.
Fossdal et al. (2007) Physiol. Mol. Plant. Pathol. 70: 161-173.
Fujimura et al. (1985) Plant Tissue Cultural Letters 2: 74.
Fujita et al. (2004) Plant J. 39: 863-876.
Gao et al. (2010) Plant Cell Tissue Organ Culture 100: 255-262.
Gotor et al. (1993) Plant J. 3: 509-518.
Graham et al. (1973) Virology 54: 536-539.
Grant et al. (1995) Plant Cell Rep. 15:254-258.
Hao et al. (2010) Planta 232: 1033-1043.
Harayama (1998) Trends Biotechnol. 16: 76-82.
He et al. (2005) Plant J. 44: 903-916.
Hegedus et al. (2003) Plant Mol. Biol. 53: 383-397.
Henikoff et al. (2004) Plant Physiol 135: 630-636.
Himi et al., (2002) J Exp Bot 53: 1569-1574.
Hinchee et al. (1988) Biotech. 6: 915.
Hsu et al. (2007) Planta 226: 311-322.
Hu et al. (2006) Proc. Natl. Acad. Sci. USA 103: 12987-12992.
Hu et al. (2008) Plant Mol. Biol. 67: 169-181.
Huang et al. (2000) Plant Cell Physiol. 41: 477-485.
Hulbert et al. (2007) Phytopathology 97: 1083-1093.
Irizarry et al. (2003) Biostatistics 4: 249-264.
Jensen et al. (2010) Biochem. J. 426: 183-196.
Jeong et al. (2009) Plant Biotechnol. Rep. 3: 127-134.
Jeong et al. (2010) Plant Physiol. 153: 185-197.
Jordan et al. (2007) Plant Biotech. J. 5: 442-453.
Joshi (1987) Nucl. Acid Res. 15: 6643-6653.
Kam et al. (2008) Plant Mol. Biol. 67: 305-322.
Karaba et al. (2007) Proc. Natl. Acad. Sci. U.S.A. 104: 15270-15275.
Kato et al., (2001) Theor Appl Genet. 102: 980-985.
Kikuchi et al. (2000) Mol. Gen. Genet. 262: 1047-1051.
Kim et al. (2006) Plant Cell 18: 3132-3144.
Kim et al. (2007) J. Biol. Chem. 282: 36292-36302.
Kwon et al. (1994) Plant Physiol. 105: 357-367.
Langridge et al., (2001) Aust J Agric Res 52: 1043-1077
Li et al. (2004). Functional Integrat Genomics 4: 231-240.
Liu et al. (1998) Plant Cell 10:1391-1406.
Lu et al. (1993) J. Exp. Med. 178: 2089-2096.
Maas et al. (1997) Mol. Breeding. 3: 15-28.
Mares et al. (2005). Theor Appl Genet Epub ahead of print.
Matsuoka et al. (1993) Proc. Natl. Acad. Sci. USA 90: 9586-9590.
Mauch-Mani and Flors (2009) Cell Res. 19: 1322-1323.
McElroy et al. (1990) Plant Cell 2:163-171.
Medberry et al. (1992) Plant Cell 4: 185-192.
Medberry et al. (1993) Plant J. 3: 619-626.
Mori et al., (2005). Theor Appl Genet. 110: 1315-1323.
Mott and Wang (2007) Plant Sci. 173: 327-339.
Nakamura et al. (2008) Plant Physiol. 147:391-401.
Nakashima et al. (2007) Plant J. 51: 617-630.
Needleman and Wunsch (1970) J. Mol. Biol. 45: 443-453.
Niedz et al. (1995) Plant Cell Reports 14: 403-406.
Nuruzzaman et al. (2010) Gene 465: 30-44.
Odell et al. (1985) Nature 313:810-812.
Orozco et al. (1993) Plant Mol. Biol. 23: 1129-1138.
Ow et al. (1986) Science 234: 856-859.
Patel et al. (2000) Mol. Breeding. 6: 113-124.
Pellegrineschi et al. (2002) Genome 45: 421-430.
Peng et al. (2010) Mol. Biotechnol. 44: 30-40.
Pfaffl (2001) Nucl. Acids Res. 29: e45.
Pinheiro et al. (2009) Gene 444: 10-23.
Prada et al. (2004). Theor Appl Genet. 109: 62-70.
Prasher et al. (1985) Biochem. Biophys. Res. Comm. 126: 1259-68.
Rae (2007) Annu. Rev. Plant Biol. 58: 347-375.
Robertson (2004) Plant Physiol. 136: 2747-2761.
Ruiter et al. (1997) Plant Mol. Biol. 34: 163-168.
Sablowski et al. (1998) Cell 92: 93-103.
Sharp et al., (2001) Aust J Agric Res 52: 1357-1366
Shaw et al. (2009) Funct. Integr. Genomics. 9: 485-498.
Shewmaker et al., (1999) Plant J 20: 401-412
Singla-Pareek et al. (2003) Proc. Natl. Acad. Sci. U.S.A. 10: 14672-14677.
Skirycz et al. (2011) Nature Biotech. 29:212-214.
Slade and Knauf (2005) Transgenic Res. 14: 109-115.
Souer et al. (1996) Cell 85: 159-170.
Stalker et al. (1988) Science 242: 419-423.
Stepansky and Galili. (2003) Plant Physiol. 133: 1407-1415.
Stephenson et al. (2007) Plant Mol. Biol. 65: 77-92.
Stephenson et al. (2010) Funct. Integr. Genomics 10: 265-276.
Stockhaus et al. (1987) Proc. Natl. Acad. Sci. USA 84: 7943-7947.
Stockhaus et al. (1989) EMBO J. 8: 2445-2451.
Takada et al. (2001) Develop. 128: 1127-1135.
Takasaki et al. (2010) Mol. Genet. Genomics 284: 173-183.
Tateishi et al. (2001) Biosci. Biotechnol. Biochem. 65: 1663-1668.
Thillet et al. (1988) J. Biol. Chem. 263: 12500.
Toriyama et al. (1986) Theor. Appl. Genet. 205: 34.
Tran et al. (2004) Plant Cell 16: 2481-2498.
Tran et al. (2010) GM Crops 1: 32-39.
Uaury et al. (2006) Science 314: 1298-1301.
Velten et al. (1984) EPBO J. 3:2733-2730.
Vilardell et al. (1990) Plant Mol Biol 14:423-432.
Wagner et al. (1992) Proc. Natl. Acad. Sci. USA 89: 6099-6103.
Wan et al. (2008) BMC Genomics 9: 121.
Watanabe et al. (2010) FEBS Lett. 584: 1181-1186.
Xia et al. (2010a) Physiol. Mol. Plant. Pathol. 74: 394-402.
Xia et al. (2010b) Mol. Biol. Rep. 37: 3703-37U.
Xiao and Xue (2001) Plant Cell Rep. 20: 667-673.
Xiao et al. (2009) Mol. Plant. 2: 73-83.
Xie et al. (1999) Plant Mol. Biol. 39: 647-656.
Xie et al. (2000) Genes Dev. 14: 3024-3036.
Xie et al. (2002) Nature 419: 167-170.
Xue (2002) Nucl. Acids Res. 30: e77.
Xue (2005) Plant J. 41: 638-649.
Xue and Loveridge (2004) Plant J. 37: 326-339.
Xue et al. (2006) Func. Plant Biol. 33: 43-57.
Xue et al. (2008b) Plant Physiol. 146: 441-454.
Xue et al. (2008a) Plant Mol. Biol. 67: 197-214.
Yadav et al. (2005) Biochem. Biophys. Res. Comm. 337: 61-67.
Yamamoto et al. (1994) Plant Cell Physiol. 35: 73-778.
Yamamoto et al. (1997) Plant J. 1: 255-265.
Yang et al. (2010) Mol. Plant. 3: 469-490.
Yeh et al. (2000) Plant Physiol. 124: 1251-1265.
Zhao et al. (2010) Plant J. 63: 100-114.
Zheng et al. (2009) Biochem. Biophys. Res. Commun. 379: 985-989.
Zhong et al. (2007) Planta 225: 1603-1611.

1. A transgenic wheat plant comprising an exogenous polynucleotide encoding a polypeptide comprising amino acids having a sequence as provided in any one of SEQ ID NOs: 1 to 4, a biologically active fragment thereof, or an amino acid sequence which is at least 90% identical to any one or more of SEQ ID NOs: 1 to 4, wherein the polypeptide is a NAC transcription factor, and wherein the polynucleotide is operably linked to a promoter capable of directing expression of the polynucleotide in a cell of the plant, and wherein the plant is more tolerant to stress conditions than an isogenic wheat plant lacking the exogenous polynucleotide.

2. The plant of claim 1, wherein
i) the promoter is a plant stress inducible promoter, and/or
ii) the NAC transcription factor is produced in at least a part of the plant at a level at least about 5 fold or 10 fold or 15 fold or 20 fold higher in the transgenic plant when compared to an isogenic wheat plant lacking the exogenous polynucleotide.

3. The plant of claim 1, which when grown under stress conditions
i) produces at least about 1.25 fold or 1.5 fold or 1.75 fold or 2 fold greater shoot and/or root biomass when compared to an isogenic wheat plant lacking the exogenous polynucleotide,
ii) has greater root length when compared to an isogenic wheat plant lacking the exogenous polynucleotide,
iii) has a better water use efficiency than an isogenic wheat plant lacking the exogenous polynucleotide,
iv) has a greater seed yield when compared to an isogenic wheat plant lacking the exogenous polynucleotide,
v) has enhanced expression of an endogenous gene when compared to an isogenic wheat plant lacking the exogenous polynucleotide, wherein the endogenous gene encodes: xyloglucan endo-transglycosylase, dioxygenase, HPPDase, glyoxalase Ia, glyoxalase Ib, chitinase 3-like, xanthine dehydrogenase, beta-alanine synthase, a ZIM family gene, chitinase 3, wali3-like, saccharopine dehydrogenase-like, or cellulose synthase-like protein CslE, or
vi) has a combination of two or more of the features of i) to v).

4. The plant according to claim 1, which, when grown under non-stress conditions, produces about the same seed yield, number and/or weight as an isogenic wheat plant lacking the exogenous polynucleotide.

5. The plant according to claim 1, wherein the stress condition is water limitation, salt stress, heat stress, cold stress, fungal infection, or a combination of two or more thereof.

6. The plant according to claim 1 which is of the species Triticum aestivum ssp aestivum or Triticum durum.

7. The plant according to claim 1 which is homozygous for the exogenous polynucleotide.

8. The plant according to claim 1 which is growing in a field.

9. A population of at least 100 wheat plants according to claim 1 growing in a field.

10. An isolated and/or exogenous polynucleotide comprising nucleotides having
i) a sequence as provided in any one of SEQ ID NOs: 5 to 8,
ii) a sequence which is at least 90% identical to any one or more of SEQ ID NOs: 5 to 8, or
iii) a sequence which encodes a polypeptide comprising amino acids having a sequence as provided in any one of SEQ ID NOs: 1 to 4, a biologically active fragment thereof, or an amino acid sequence which is at least 90% identical to any one or more of SEQ ID NOs: 1 to 4,
wherein the polynucleotide encodes a NAC transcription factor, and wherein the polynucleotide is operably linked to a promoter capable of directing expression of the polynucleotide in a cell of a wheat plant.

11. (canceled)

12. A vector comprising the polynucleotide of claim 10.

13. A cell comprising the polynucleotide of claim 10.

14. (canceled)

15. A method of producing a transgenic wheat plant according to claim 1, the method comprising the steps of
i) introducing the polynucleotide of claim 10 into a cell of a wheat plant,
ii) regenerating a transgenic plant from the cell, and
iii) optionally producing one or more progeny from the transgenic plant,
thereby producing the transgenic wheat plant.

16. A method of producing a transgenic wheat plant according to claim 1, the method comprising the steps of
i) crossing two parental wheat plants, wherein at least one is a transgenic wheat plant according to claim 1,
ii) screening one or more progeny plants from the cross for the presence or absence of the exogenous polynucleotide, and
iii) selecting a progeny plant which comprise the exogenous polynucleotide,
thereby producing the transgenic wheat plant.

17-24. (canceled)

25. A method of producing seed, the method comprising;
i) growing a wheat plant according to claim 1,
ii) harvesting the seed from the plant, and
iii) optionally processing the seed into a product which is not capable of germinating.

26-28. (canceled)

29. A seed of a plant according to claim 1.

30. (canceled)

30. A method of producing flour, wholemeal, starch or other product, comprising;
a) obtaining seed of claim 29, and
b) processing the seed to produce the flour, wholemeal, starch or other product.

31. A product produced from a plant according to claim 1, or a part thereof.

32-33. (canceled)

34. The product of claim 1, wherein the food product is selected from the group consisting of: flour, starch, leavened or unleavened breads, pasta, noodles, animal fodder, breakfast cereals, snack foods, cakes, malt, beer, pastries and foods containing flour-based sauces.

35-40. (canceled)

41. The plant of claim 2, which when grown under stress conditions
i) produces at least about 1.25 fold or 1.5 fold or 1.75 fold or 2 fold greater shoot and/or root biomass when compared to an isogenic wheat plant lacking the exogenous polynucleotide,
ii) has greater root length when compared to an isogenic wheat plant lacking the exogenous polynucleotide,
iii) has a better water use efficiency than an isogenic wheat plant lacking the exogenous polynucleotide,
iv) has a greater seed yield when compared to an isogenic wheat plant lacking the exogenous polynucleotide,
v) has enhanced expression of an endogenous gene when compared to an isogenic wheat plant lacking the exogenous polynucleotide, wherein the endogenous gene encodes: xyloglucan endo-transglycosylase, dioxygenase, HPPDase, glyoxalase Ia, glyoxalase Ib, chitinase 3-like, xanthine dehydrogenase, beta-alanine synthase, a ZIM family gene, chitinase 3, wali3-like, saccharopine dehydrogenase-like, or cellulose synthase-like protein CslE, or
vi) has a combination of two or more of the features of i) to v).
